(19) United States
(12) Patent Application Publication
(10) Pub. No.: US 2024/0226155 A1
(43) Pub. Date: Jul. 11, 2024

(54) Methods and Products for Reducing HIV Reservoirs

(71) Applicant: The Regents of the University of California, Oakland, CA (US)

(72) Inventors: Jocelyn Kim, Encino, CA (US); Jerome A. Zack, Tarzana, CA (US)

(21) Appl. No.: 18/393,997

(22) Filed: Dec. 22, 2023

Related U.S. Application Data
(60) Provisional application No. 63/437,883, filed on Jan. 9, 2023.

(51) Int. Cl. A61K 35/17; A61K 31/366; A61P 31/18

(52) U.S. Cl. CPC A61K 35/17; A61K 31/366; A61P 31/18

(57) ABSTRACT

Disclosed herein are compositions, kits, combination products, and methods that comprise a latency reversing agent such as a bryostatin compound, e.g., SUW133, in combination with NK cells for reducing amounts of a human immunodeficiency virus (HIV) in subjects.

### CROSS-REFERENCE TO RELATED APPLICATIONS

This application claims the benefit of U.S. Patent Application No. 63/437,883, filed Jan. 9, 2023, which is herein incorporated by reference in its entirety.

### ACKNOWLEDGEMENT OF GOVERNMENT SUPPORT

This invention was made with Government support under AI124743, AI131294, AI155232, awarded by the National Institutes of Health. The Government has certain rights in the invention.

### BACKGROUND OF THE INVENTION

### 1. Field of the Invention

The field of the invention generally relates to methods and compositions for treating HIV infection.

### 2. Description of the Related Art

HIV is difficult to eradicate due to the persistence of a long-lived reservoir of latently infected cells. Previous studies have shown that natural killer cells inhibit HIV infection, but it is unclear whether the administration of natural killer cells can reduce rebound viremia when anti-retroviral therapy is discontinued.

Even though anti-retroviral therapy (ART) can effectively halt HIV replication, ART must be maintained for life because latent HIV-1 infected cells persist and initiate active virus replication if ART is discontinued. Experimental approaches for eliminating the latent reservoir have included myeloablation followed by transplantation of cells lacking co-receptors for virus infection, genome editing of the latent provirus, or use of viral inducers in the presence of ART. Various latency reversing agents (LRAs) have been used to kick (or shock) infected cells out of latency and induce their death by virus-induced cytopathic effects, but a kick alone may not be sufficient in eliminating the HIV reservoir. Kill approaches have included immunological therapies that target HIV-infected cells by enhancing endogenous anti-viral immune responses or harnessing of antibody-based or effector cell therapies. One combinatorial kick and kill approach comprised of multiple LRAs combined with broadly neutralizing antibodies (bNAbs) 3BNC117, 10-1074, and PG16 administered during ART decreased viral rebound after ART interruption in humanized mice infected with HIV-1. Other kick and kill approaches utilizing a TLR7 agonist and Ad26/MVA vaccine or bNAb PGT121, decreased viral rebound after ART interruption in rhesus monkeys infected with SHIV. These preclinical studies demonstrate the enhancement of endogenous antiviral immunity or addition of bNAbs as a dedicated kill agent to LRAs diminish viral rebound from the reservoir.

Unfortunately, clinical trials employing kick and kill approaches against HIV have yet to yield promising results.

### SUMMARY OF THE INVENTION

In some embodiments, the present invention is directed to compositions, kits, and combination products for reducing an amount of a human immunodeficiency virus (HIV), e.g., HIV-1 or HIV-2, in a subject, which comprise, consist essentially of, or consist of a bryostatin compound in combination with natural killer (NK) cells. In some embodiments, the bryostatin compound, is SUW133. In some embodiments, the NK cells are CD56⁺CD3⁻ NK cells. In some embodiments, the NK cells are human peripheral blood NK cells. In some embodiments, the composition, the kit, or the combination product comprises about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, or about 120 mg of the bryostatin compound; and about 60×10⁶, about 90×10⁶, about 120×10⁶, about 150×10⁶, about 180×10⁶, about 210×10⁶, about 240×10⁶, about 270×10⁶, about 300×10⁶, about 330×10⁶, about 360×10⁶, about 390×10⁶, about 420×10⁶, about 450×10⁶, about 480×10⁶, about 510×10⁶, about 540×10⁶, about 570×10⁶, about 600×10⁶, about 630×10⁶, about 660×10⁶, about 690×10⁶, about 720×10⁶, about 750×10⁶, about 780×10⁶, about 810×10⁶, about 840×10⁶, about 870×10⁶, about 900×10⁶, about 930×10⁶, about 960×10⁶, about 990×10⁶, about 1020×10⁶, about 1050×10⁶, about 1080×10⁶, about 1110×10⁶, about 1140×10⁶, about 1170×10⁶, about 1200×10⁶, about 1230×10⁶, about 1260×10⁶, about 1290×10⁶, about 1320×10⁶, about 1350×10⁶, about 1380×10⁶, about 1410×10⁶, about 1440×10⁶, about 1470×10⁶, about 1500×10⁶, about 1530×10⁶, about 1560×10⁶, about 1590×10⁶, about 1620×10⁶, about 1650×10⁶, about 1680×10⁶, about 1710×10⁶, about 1740×10⁶, about 1770×10⁶, or about 1800×10⁶ of the NK cells.

In some embodiments, the present invention is directed to methods of reducing an amount of a human immunodeficiency virus (HIV), e.g., HIV-1 or HIV-2, in a subject, which comprise, consist essentially of, or consist of administering to the subject a bryostatin compound and NK cells. In some embodiments, the bryostatin compound, is SUW133. In some embodiments, the NK cells are CD56⁺CD3⁻ NK cells. In some embodiments, the NK cells are human peripheral blood NK cells. In some embodiments, the NK cells are administered about 2-14 hours, about 4-12 hours, about 6-10 hours, or about 8 hours after administration of the bryostatin compound. In some embodiments, the bryostatin compound is administered 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, or 24 days after ART cessation. In some embodiments, the amount of the bryostatin compound administered to the subject is about 0.75-2, about 1.0-1.75, about 1.1-1.3, about 1.25 mg/kg body weight of the subject; or about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, or about 120 mg. In some embodiments, the amount of the NK cells administered to the subject is about 1-30×10⁶, about 10-30×10⁶, or about 25×10⁶ of NK cells/kg body weight of the subject; or about 60×10⁶, about 90×10⁶, about 120×10⁶, about 150×10⁶, about 180×10⁶, about 210×10⁶, about 240×10⁶, about 270×10⁶, about 300×10⁶, about 330×10⁶, about 360×10⁶, about 390×10⁶, about 420×10⁶, about 450×10⁶, about 480×10⁶, about 510×10⁶, about 540×10⁶, about 570×10⁶, about 600×10⁶, about 630×10⁶, about 660×10⁶, about 690×10⁶, about 720×10⁶, about 750×10⁶, about 780×10⁶, about 810×10⁶, about 840×10⁶, about 870×10⁶, about 900×10⁶, about 930×10⁶, about 960×10⁶, about 990×10⁶, about 1020×10⁶, about 1050×10⁶, about 1080×10⁶, about 1110×10⁶, about 1140×10⁶, about 1170×10⁶, about 1200×10⁶, about 1230×10⁶, about 1260×10⁶, about 1290×10⁶, about 1320×10⁶, about 1350×10⁶, about 1380×10⁶, about 1410×10⁶, about 1440×10⁶, about 1470×10⁶, about 1500×10⁶, about 1530×10⁶, about 1560×10⁶, about 1590×10⁶, about 1620×10⁶, about 1650×10⁶, about 1680×10⁶, about 1710×10⁶, about 1740×10⁶, about 1770×10⁶, or about 1800×10⁶. In some embodiments, the amount of the bryostatin compound administered to the subject is about 0.75-2, about 1.0-1.75, about 1.1-1.3, about 1.25 mg/kg body weight of the subject of bryostatin compound; and the amount of the NK cells administered to the subject is about 1-30×10⁶, about 10-30×10⁶, or about 25×10⁶ of the NK cells/kg body weight of the subject. In some embodiments, the amount of the bryostatin compound administered to the subject is about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, or about 120 mg; and the amount of the NK cells administered to the subject is about 60×10⁶, about 90×10⁶, about 120×10⁶, about 150×10⁶, about 180×10⁶, about 210×10⁶, about 240×10⁶, about 270×10⁶, about 300×10⁶, about 330×10⁶, about 360×10⁶, about 390×10⁶, about 420×10⁶, about 450×10⁶, about 480×10⁶, about 510×10⁶, about 540×10⁶, about 570×10⁶, about 600×10⁶, about 630×10⁶, about 660×10⁶, about 690×10⁶, about 720×10⁶, about 750×10⁶, about 780×10⁶, about 810×10⁶, about 840×10⁶, about 870×10⁶, about 900×10⁶, about 930×10⁶, about 960×10⁶, about 990×10⁶, about 1020×10⁶, about 1050×10⁶, about 1080×10⁶, about 1110×10⁶, about 1140×10⁶, about 1170×10⁶, about 1200×10⁶, about 1230×10⁶, about 1260×10⁶, about 1290×10⁶, about 1320×10⁶, about 1350×10⁶, about 1380×10⁶, about 1410×10⁶, about 1440×10⁶, about 1470×10⁶, about 1500×10⁶, about 1530×10⁶, about 1560×10⁶, about 1590×10⁶, about 1620×10⁶, about 1650×10⁶, about 1680×10⁶, about 1710×10⁶, about 1740×10⁶, about 1770×10⁶, or about 1800×10⁶.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description explain the principles of the invention.

### DESCRIPTION OF THE DRAWINGS

This invention is further understood by reference to the drawings wherein:

FIG. 1 to FIG. 8. NK cells delay time to viral rebound after ART interruption in BLT mice infected with HIV strain NFNSX. FIG. 1, Schematic representation of experiment; acute infection of TKO-BLT mice with NFNSX for four weeks, ART for six weeks. One day after ART interruption, 5×10⁶ allogeneic human peripheral blood NK cells were transferred followed by another homologous dose of NK cells five days later. The red arrows denote when NK cells were given. FIG. 2, Longitudinal plasma viral loads for each infected animal at various timepoints in the No NK and NK groups. Gray shading indicates ART treatment period of six weeks. Dashed line starting that −2 weeks indicates the median frequency of CD56⁺CD3⁻ cells detected in the blood. FIG. 3, Kaplan-Meier curves showing the frequency of aviremic mice after ART interruption. P value calculated by log-rank Mantel-Cox test. FIG. 4, Frequency of CD56⁺CD3⁻ cells in the blood five days after the first injection of NK cells. FIG. 5, FIG. 6, Plasma viral loads for each animal before ART was initiated (FIG. 5) and at necropsy (FIG. 6). FIG. 7, FIG. 8, Cell-associated “CA” HIV RNA (FIG. 7) and HIV DNA (FIG. 8) levels from the spleen and bone marrow of each infected animal. n=6 biologically independent animals in each group observed over one independent experiment (FIG. 2 to FIG. 8). Black dotted line indicates the detection limit of 2.3 log RNA copies per ml (FIG. 2, FIG. 5, FIG. 6) and 1.0 log RNA or DNA copies (FIG. 7, FIG. 8). Horizontal bars represent the means (FIG. 4 to FIG. 8). P values were calculated using two-tailed Mann-Whitney test (FIG. 4 to FIG. 8). Source data are provided as a Source Data file.

FIG. 9 to FIG. 14. NK cells delay viral rebound after ART interruption. FIG. 9, 21 bp barcode region constrained by a thymine every 3^{rd} nt was inserted upstream of the hemagglutinin “HA” tag in a non-functional vpr of the HIV strain NL-HA. FIG. 10, Schematic representation of experiment; acute infection of NSG-BLT with NL-HABC. FIG. 11, Longitudinal plasma viral loads for each infected animal at various timepoints in the No NK (top) and NK (bottom) groups. Gray shading indicates ART treatment period of six weeks. Dashed line starting at time 0 indicates the median frequency of CD56⁺CD3⁻ cells detected in the blood. FIG. 12, Kaplan-Meier curves showing the frequency of aviremic mice after ART interruption. P value denotes statistically significant difference calculated by log-rank Mantel-Cox test. FIG. 13, Frequency of CD56⁺CD3⁻ cells in the blood five days after the first injection of NK cells among mice that rebounded “Reb.” (squares) or did not rebound “No Reb.” (circles) after ART discontinuation. FIG. 14, Plasma viral loads for each animal before ART was initiated. n=12 biologically independent animals in No NK group, n=14 biologically independent animals in NK group (FIG. 11, FIG. 12). n=18 biologically independent animals in Reb. group, n=8 biologically independent animals in No Reb. group (FIG. 13, FIG. 14). Black dotted line indicates the detection limit of 2.3 log RNA copies per ml (FIG. 11, FIG. 14). Horizontal bars represent the means (FIG. 13, FIG. 14). P values were calculated using two-tailed Mann-Whitney test (FIG. 13, FIG. 14). Results are pooled from two independent experiments. Source data are provided as a Source Data file.

FIG. 15 to FIG. 18. Viral RNA and DNA levels in NL-HABC-infected BLT mice treated with NK cells. FIG. 15, Plasma viral RNA levels for each animal before ART was initiated in the No NK (squares) and NK (circles) groups. FIG. 16, Plasma viral load at necropsy of the animals that rebounded after ART interruption. FIG. 17, FIG. 18, Cell-associated HIV RNA (FIG. 17) or DNA (FIG. 18) is shown from the spleen and bone marrow “BM” of mice that were aviremic “A” or had rebound viremia “V” at necropsy. n=12 biologically independent animals in No NK group, n=14 biologically independent animals in NK group (FIG. 15, FIG. 17, FIG. 18). Among the rebounding animals, n=12 biologically independent animals in No NK group, n=6 biologically independent animals in NK group (FIG. 16). Black dotted line indicates the detection limit of 2.3 log RNA copies per ml (FIG. 15, FIG. 16) or 1.0 log RNA or DNA copies (FIG. 17, FIG. 18). Horizontal bars represent the means. P values were calculated using two-tailed Mann-Whitney test. Source data are provided as a Source Data file.

FIG. 19 to FIG. 20. NK cells decrease barcode diversity in NL-HABC infected mice. Stacked barplots displaying the relative frequencies and distribution of major barcode variants identified in the plasma, spleen and bone marrow “BM” of mice treated with or without NK cells during ART interruption described in FIG. 9 to FIG. 14 were generated. FIG. 19, Number of unique barcodes quantified by deep sequencing of viral RNA from pooled organs per mouse in the No NK (squares) and NK (circles) groups. n=12 biologically independent animals in No NK group, n=14 biologically independent animals in NK group. FIG. 20, Number of unique barcodes from the plasma (n=11 biologically independent animals in no NK group, 13 biologically independent animals in NK group), spleen and BM (n=12 biologically independent animals in no NK group, n=14 biologically independent animals in NK group) of each mouse. Horizontal bars represent the means. P values were calculated using two-tailed Mann-Whitney test. Source data are provided as a Source Data file.

FIG. 21 to FIG. 24. SUW133 plus NK cells delays viral rebound after ART interruption. FIG. 21, Schematic representation of experiment; acute infection of TKO-BLT mice with NL-HABC. ART for six weeks. SUW 133 or control vehicle DMSO was administered five days prior to stopping ART. 5×10⁶ allogeneic human peripheral blood NK cells were injected the same day as injection of SUW133 or DMSO and another homologous dose of cells were given five days later on the day of ART discontinuation. FIG. 22, Longitudinal plasma viral loads for each infected animal at various timepoints in the DMSO only, NK only, LRA only, and LRA plus NK groups. Gray shading indicates ART treatment period of 4.7 weeks. Dashed line starting at −1 week indicates the median frequency of CD56⁺CD3⁻ cells detected in the blood. Black dotted line indicates the detection limit of 2.3 log RNA copies per ml. FIG. 23, Kaplan-Meier curves showing frequency of aviremic mice after ART interruption. FIG. 24, Frequency of CD56⁺CD3⁻ cells in the blood five days after the first injection of NK cells. Horizontal bars represent the means P values were calculated using two-tailed Mann-Whitney test. n=11 biologically independent animals in DMSO group, n=5 biologically independent animals in NK group, n=8 biologically independent animals in LRA group, n=10 biologically independent animals in LRA plus NK group (FIG. 22 to FIG. 24). Results are pooled from two independent experiments. Source data are provided as a Source Data file.

FIG. 25 to FIG. 30. Levels of viral RNA, barcode diversity, and HIV DNA from infected mice treated with SUW133 and NK cells. FIG. 25, Plasma viral loads of rebounding mice. n=11 biologically independent animals in DMSO (squares) group, n=5 biologically independent animals in NK only (circles) group, n=8 biologically independent animals in LRA only (point up triangles) group, n=6 biologically independent animals in LRA plus NK (point down triangles) group. FIG. 26, Cell-associated “CA” HIV RNA from the spleen and bone marrow “BM” of mice that were aviremic “A” or had rebound viremia “V” at necropsy. FIG. 27, FIG. 28, Number of unique barcodes quantified by deep sequencing of viral RNA from pooled organs per mouse (FIG. 27) or from the plasma, spleen, and BM of each mouse (FIG. 28). FIG. 29, Scatterplot of number of unique barcodes per mouse and time to rebound among all mice that rebounded after ART interruption. n=48 biologically independent rebounding animals. Lines are linear predictions of time to rebound on number of barcodes. The 95% confidence intervals of the fitted values are shown by grey areas. r=Pearson correlation coefficient. FIG. 30, Total CA HIV DNA from the spleen and BM of mice that were aviremic or had rebound viremia at necropsy. n=11 biologically independent animals in DMSO only group, n=5 biologically independent animals in NK only group, n=8 biologically independent animals in LRA only group, n=10 biologically independent animals in LRA plus NK group (FIG. 26 to FIG. 28, FIG. 30). Black dotted line indicates the detection limit of 2.3 log RNA copies per ml (a) or 1.0 log RNA or DNA copies (FIG. 26, FIG. 30). Horizontal bars represent the means (FIG. 25 to FIG. 28, FIG. 30). P values were calculated using one-way ANOVA Kruskal-Wallis test (FIG. 25, FIG. 26, FIG. 30), two-tailed Mann-Whitney (FIG. 27, FIG. 28), or Pearson correlation test (FIG. 29). Results are pooled from two independent experiments. Source data are provided as a Source Data file.

FIG. 31 to FIG. 36. Allogeneic NK cells were effective at inhibiting the R5-tropic NFNSX in vitro and in vivo. NK cells are activated by HIV-infected CD4 T cells and inhibit HIV replication. FIG. 31, Experimental design (top) of allogeneic peripheral NK cells and CD4 T cells. Representative FACS plots of IFN-γ and CD107a expression on the CD3″ gated NK cells when cocultured for 24 h with NL4-3 infected or uninfected CD4 T cells at a ratio of 1:1. FIG. 32, Frequency of allogeneic NK cells expressing IFN-γ and CD107a when cocultured with NL4-3, NFNSX-infected, or uninfected CD4 T cells at 1:1 ratio for 24 h. Positive control NK cells were activated with PMA and ionomycin (PMA/lo). n=6 independent biologically replicates from three independent experiments. FIG. 33, FIG. 34, Frequency of IFN-γ and CD107a (FIG. 33) or HIV gag p24 (FIG. 34) expressing NK cells that were exposed to varying doses of cell-free NL4-3 or NFNSX supernatant or cell media without virus (Uninf.) or with PMA and ionomycin (PMA/lo) in the absence of CD4⁺ T cells. FIG. 35, FIG. 36, Representative FACS plots (FIG. 35) and graph (FIG. 36) showing the frequency of p24⁺ CD4⁺ T cells that were infected with NL4-3, NFNSX, or uninfected (Uninf.) when cultured with or without allogeneic peripheral NK cells at 1:1 ratio for 48 h. n=3 independent biologically replicates from one representative experiment. Shown are mean f SEM (FIG. 32, FIG. 33, FIG. 34). P values were calculated using two-tailed paired t-test (FIG. 32, FIG. 36) or one-way ANOVA (FIG. 33, FIG. 34). Source data are provided as a Source Data file.

FIG. 37 to FIG. 41. Human immune engraftment of NFNSX-infected BLT mice treated with NK cells. FIG. 37, Frequency of human CD56+ CD3− cells in the blood, spleen, and bone marrow “BM” at necropsy in the No NK (squares) and NK (circles) groups. FIG. 38, FIG. 39, Longitudinal frequencies and absolute counts of human CD45+ cells (FIG. 38) and CD4⁺ T cells (FIG. 39) in the blood at various timepoints. FIG. 40, FIG. 41, Frequencies and absolute counts of human CD45+ cells (FIG. 40) and CD4⁺ T cells (FIG. 41) in the spleen and BM at necropsy. n=6 biologically independent animals in each group observed over one independent experiment. Horizontal bars represent the medians. P values were calculated using two-tailed Mann-Whitney test. Source data are provided as a Source Data file.

FIG. 42 to FIG. 44. Generation and infectivity of barcoded NL-HABC. FIG. 42, HIV p24 protein levels from the virus supernatant of 293T cells transfected with plasmids encoding NL4-3 (circles) or NL-HABC (squares). Data is mean±s.d. n=4 biologically independent transfection preparations for NL4-3 and n=3 biologically independent transfection preparations for NL-HABC. FIG. 43, FIG. 44, Varying input of NL43 or NL-HABC was added to GHOST cells (FIG. 43) or co-stimulated CD4⁺ T cells (FIG. 44). Infected cells were quantified 48 h later by flow cytometry. n=3 technical replicates per group (FIG. 43). n=3 biologically independent donors per group (FIG. 44). Connecting lines indicate the median. P values were calculated using two-tailed unpaired t-test. Data are representative of one independent experiment. Source data are provided as a Source Data file.

FIG. 45 to FIG. 49. Human immune engraftment of NL-HABC-infected BLT mice treated with NK cells. Cells from mice described in the No NK (squares) and NK (circles) groups in FIG. 9 to FIG. 14 were immunophenotyped by flow cytometry. FIG. 45, Frequency of human CD56⁺ CD3⁻ cells in the blood, spleen, and bone marrow “BM” at necropsy. FIG. 46, FIG. 47, Longitudinal frequencies and absolute counts of human CD45+ cells (FIG. 46) and CD4⁺ T cells (FIG. 47) in the blood at various timepoints. FIG. 48, FIG. 49, Frequencies and absolute counts of human CD45+ cells (FIG. 48) and CD4⁺ T cells (FIG. 49) in the spleen, and BM at necropsy. n=12 biologically independent animals in No NK group, n=14 biologically independent animals in NK group Horizontal bars represent the medians. P values were calculated using two-tailed Mann-Whitney test. Source data are provided as a Source Data file.

FIG. 50. NK cells reduce viral spread after ART interruption in NL-HABC infected mice. Viral spread rate in mice that received or did not receive NK cells after ART discontinuation. The mean and the standard deviation of the experimental time-series are shown by solid lines, and linear fit by dashed lines. The best fitting equations for the HIV RNA log copies of virus (y) are provided where time (t) is measured in weeks. Converted to growth rate per day, the values are r=0.181 day⁻¹ and r=0.087 day⁻¹ for without and with NK cells, respectively. Significance of the difference between the two slopes was calculated by using z-statistics.

FIG. 51 to FIG. 54. Analysis of barcoded NL-HABC. FIG. 51, Histogram demonstrates ˜14,000 viral barcodes with relatively equal distribution as quantified by Hiseq sequencing derived from virion-associated viral RNA of the virus supernatant collected from HIV-producing transfected 293T cells. FIG. 52, Histogram shows the pair-wise distance between barcodes is 11 bp. FIG. 53, Representative histogram shows the frequency of Primer ID following a bi-modal distribution of authentic barcodes (bars right of 1.5) versus barcodes containing PCR error (bars left of 1.5). FIG. 54, RNA molecules from the same population were sampled at two different times to quantify the starting number of RNA molecules in each replicate and thereby assess the reproducibility of the results. Scatterdot plot of the two replicates (FIG. 53). r=0.92, Spearman coefficient. P=6.7×10⁻⁵. Pie chart depicting the absolute diversity of clones obtained from the same sample for each replicate (FIG. 54). Source data are provided as a Source Data file.

FIG. 55 to FIG. 57. T cell activation and correlation between pre-ART viral loads and time to rebound. FIG. 55, Frequency of CD69+CD4⁺ T cells in the blood five days after the injection of DMSO only (squares), NK only (circles), LRA only (point up triangle), and LRA plus NK (point down triangle) groups. n=10 biologically independent animals in DMSO only group, n=3 biologically independent animals in NK only group, n=8 biologically independent animals in LRA only group, n=10 biologically independent animals in LRA plus NK group. P values were calculated using two-tailed Mann-Whitney test. Mice treated with either SUW133 or SUW133 plus NK cells demonstrated a significant increase in CD69 expression on CD4⁺ T cells in vivo. FIG. 56, Plasma viral loads for each animal before ART was initiated. Black dotted line indicates the detection limit of 2.3 log RNA copies per ml. P values were calculated using one-way ANOVA Kruskal-Wallis test. n=11 biologically independent animals in DMSO only group, n=5 biologically independent animals in NK only group, n=8 biologically independent animals in LRA only group, n=10 biologically independent animals in LRA plus NK group. Horizontal bars represent the means (FIG. 55, FIG. 56). FIG. 57, Scatterplot of pre-ART HIV levels and time to rebound after ART discontinuation of the rebounding animals. Among the rebounding animals, n=11 biologically independent animals in DMSO only group, n=5 biologically independent animals in NK only group, n=8 biologically independent animals in LRA only group, n=6 biologically independent animals in LRA plus NK group. Lines are linear predictions of time to rebound on pre-ART viral loads. The 95% confidence intervals of the fitted values are shown by grey areas. r=Pearson correlation coefficient. P values were calculated using Pearson correlation test. Results are pooled from two independent experiments. Source data are provided as a Source Data file.

FIG. 58 to FIG. 62. Human immune engrafiment in infected mice treated with SUW133 and NK cells. FIG. 58, Frequency of human CD56⁺ CD3⁻ cells in the blood, spleen, and bone marrow “BM” at necropsy of the DMSO only (squares), NK only (circles), LRA only (point up triangle), and LRA plus NK (point down triangle) groups. FIG. 59, FIG. 60, Longitudinal frequencies and absolute counts of human CD45+ cells (FIG. 59) and CD4⁺ T cells (FIG. 60) in the blood at various timepoints. FIG. 61, FIG. 62, Frequencies and absolute counts of human CD45+ cells (FIG. 61) and CD4⁺ T cells (FIG. 62) in the spleen, and BM at necropsy. n=11 biologically independent animals in DMSO only group, n=5 biologically independent animals in NK only group, n=8 biologically independent animals in LRA only group, n=10 biologically independent animals in LRA plus NK group. Horizontal bars represent the medians. P values were calculated using one-way ANOVA Kruskal-Wallis test. Results are pooled from two independent experiments. Source data are provided as a Source Data file.

FIG. 63. Table summarizing the results of an ex vivo viral outgrowth assay of mice that did or did not receive NK cells. Mice from FIG. 9 to FIG. 14 were tabulated based on their treatment group, presence of rebound viremia after ART interruption and cell-associated HIV DNA in the spleens, and levels of HIV p24 antigen by ELISA of cell-free supernatant from the ex vivo viral outgrowth splenocyte cocultures at 7, 10 and 14 days after coculture. ND=not detected. Lower limit of detection was 0.25 ng of p24 per ml.

FIG. 64. Table summarizing time to rebound and number of unique barcodes in mice that received SUW133 plus NK cells. Mice from FIG. 21 to FIG. 24 were tabulated based on their experiment number, treatment group, mouse ID, time to rebound after ART interruption, and number of viral barcodes per mouse. No significant differences in the time to rebound between the DMSO groups in Experiments 4 and 5 (p=0.19, DMSO Exp1 vs DMSO Exp2) or number of barcodes (p=0.66, DMSO Exp4 vs DMSO Exp5). No significant differences in the time to rebound among rebounding animals in the SUW133 plus NK groups in Experiments 4 and 5 based (p=0.39, SUW133 plus NK Exp4 vs SUW133 plus NK Exp5) or number of barcodes (p=0.14, SUW133 plus NK Exp4 vs SUW133 plus NK Exp5). NR=no rebound. s.e.m=standard error of mean. P values were calculated using unpaired two-tailed Mann Whitney t-test.

FIG. 65. Table summarizing the results of an ex vivo viral outgrowth assay of mice that did or did not rebound after receiving SUW133 plus NK cells. Mice from FIG. 21 to FIG. 24 that received SUW133 plus NK cells and did not demonstrate presence of rebound viremia after ART interruption and cell-associated HIV DNA in the spleens, were tabulated based on levels of HIV p24 antigen by ELISA detected in the cell-free supernatant from the ex vivo viral outgrowth splenocyte cocultures at 7, 10 and 14 days after coculture. ND=not detected. Lower limit of detection was 0.25 ng of p24 per ml.

Color versions of the drawings and source data may be obtained from Kim, et al. (2022) “Latency reversal plus natural killer cells diminish HIV reservoir in vivo” Nat Commun. 13(1):121, which is herein incorporated by reference in its entirety.

### DETAILED DESCRIPTION OF THE INVENTION

As disclosed herein, the administration of allogeneic human peripheral blood natural killer (NK) cells inhibits viral rebound following interruption of anti-retroviral therapy (ART) in subjects infected with HIV. The NK cells also decreased the diversity of rebounding viral clones, and even eliminated infected cells harboring productive virus from the splenocytes of subjects infected with HIV.

A “kick and kill” strategy employing SUW133 (an analog of bryostatin, and is also a protein kinase C (PKC) modulator) as the “kick”, i.e., a latency reversing agent (LRA), and NK cells as the “kill” agent after ART interruption eliminated HIV viral reservoirs in subjects.

In the experiments herein, SUW133 was used as the LRA because a single administration activated CD4⁺ T cells and reversed latency in vivo. However, any LRA known in the art may be employed. In addition to SUW133, suitable latency reversing agents (LRAs) include histone deacetylase inhibitors (e.g., Trichostatin A, trapoxin, suberoylanilide hydroxamic acid, romidepsin, vorinostat, panobinostat, entinostat, valproic acid, fimepinostat, and chidamide); histone methyltransferase inhibitors (e.g., Chaetocin and BIX-01294); DNA methylation inhibitors (e.g., 5-aza-cytidine, 5-aza-deoxycytidine, and zebularine); PKC agonists (e.g., Phorbol 12-myristate 13-acetate, prostratin, SUW013 and C13 analogs, bryostatin compounds (e.g., byrostatin-1 to -20, SUW133), and ingenol, and analogs thereof, BET inhibitors (e.g., JQ1, I-BET, I-BET151, MMQO, RVX-208, and PFI-1); TLR agonists (e.g., Flagellin, Pam3CSK4, GS-9620, and MGN1703); activators of the Akt pathway (e.g., Disulfiram and hexamethylene bisacetamide); SMAC mimetics (e.g., SBI-0637142, SBI-0953294, and AZD5582); and STAT5 sumoylation inhibitors (e.g., 1-hydroxybenzotriazol, 1-hydroxy-7-amino benzotriazole, and 3-hydroxy-1,2,3-benzotriazin-4(3H)-one and analogs).

It is important to note that in prior studies, SUW133 alone delayed rebound viremia for up to two to four weeks after ART interruption, but the splenocytes from the SUW133-treated mice generated replication-competent virus in ex vivo cultures, thereby indicating that all SUW133-treated mice would have eventually rebounded. In the experiments herein, viral loads were monitored for about twelve weeks after ART interruption, and thereby confirms that viral loads in subjects receiving SUW133 alone (without NK cells) eventually rebound. Additionally, prior studies employed NSG-BLT mice. However, in the experiments herein, TKO-BLT mice, which have lower levels of endogenous immune activation and are therefore better models of HIV immunocompromised subjects were employed.

The timing of NK cellular injections may also play a role in the kick and kill approach disclosed herein. The diversity of barcodes of rebounding viruses were reduced with NK cell treatment, which indicates that NK cells inhibit or eliminate the reactivating latently infected T cells before viral propagation to new target cells can be completed. Thus, the window of activity of NK cells might be after activation of T cells by LRA and before viral replication occurs. This may explain why NK cells were effective when administered after ART interruption. In contrast, NK cells alone that were administered while virus replication was suppressed on ART failed to significantly delay viral rebound, presumably because NK cells do not act as a kick to increase HIV-expressing target cells in vivo. It is also possible that the diminished basal immune activation state of TKO-BLT mice may have reduced the likelihood that NK cells would recognize any spontaneously activating latently infected cells on ART.

### NK Cells are Activated by Allogeneic HIV-Infected CD4⁺ T Cells

Whether peripheral blood NK cells could be utilized as an efficient kill agent against cells productively infected with HIV was investigated. Human peripheral blood NK cells from the peripheral blood mononuclear cells (PBMCs) of four healthy donors were obtained using magnetic bead isolation. Uniform Manifold Approximation and Projection (UMAP) was used to visualize subpopulations among the CD56⁺CD3⁻ cells from concatenated and individual samples by flow cytometry (data not shown). Intensity of the expression of markers was visualized using a rainbow heat scale. Manually assigned clusters were assigned on the concatenated sample. Histograms of surface markers were generated with x-axis measuring marker intensity and y-axis normalized to mode, and frequencies of manually assigned clusters among CD56⁺CD3⁻ cells across individual donor samples was obtained. Clusters were manually assigned based on the differential expression of eleven surface markers and five subpopulations, including the canonical NK cell subsets: CD56^{dim}CD16⁺ (populations 1-4) and CD56^{bright}CD16⁻ cells (population 5) were identified (data not shown). As expected, CD56^{dim}CD16⁺ NK cells were the predominant cell type in the peripheral blood (data not shown). Among the CD56^{dim}CD16⁺ NK cells, four subpopulations based on the differential expression of CD57, KIR2DL1/S1/S3/S5, and natural cytotoxicity receptor (NCR) NKp44 were identified (data not shown). In comparison to CD56^{dim}CD16⁺ NK cells, the CD56^{bright}CD16⁻ cells demonstrated increased expression of NCR NKp46 and inhibitory receptor NKG2A and decreased expression of KIR2DL1/S1/S3/S5 and CD57, which was consistent with an immature phenotype as previously reported. All subpopulations expressed high levels of the activating co-receptors 2B4 and NKp80 and cell stress sensing activating receptor NKG2D and low levels of NCR NKp30. These results are consistent with the understanding that the peripheral blood contains the classical subsets of CD56^{dim}CD16⁺ NK and CD56^{bright}CD16⁻ cells as well as additional heterogeneous subpopulations.

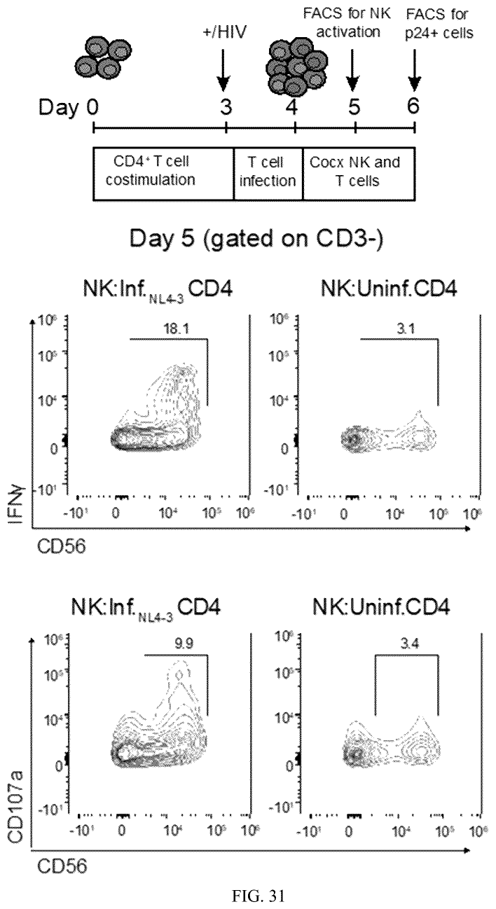

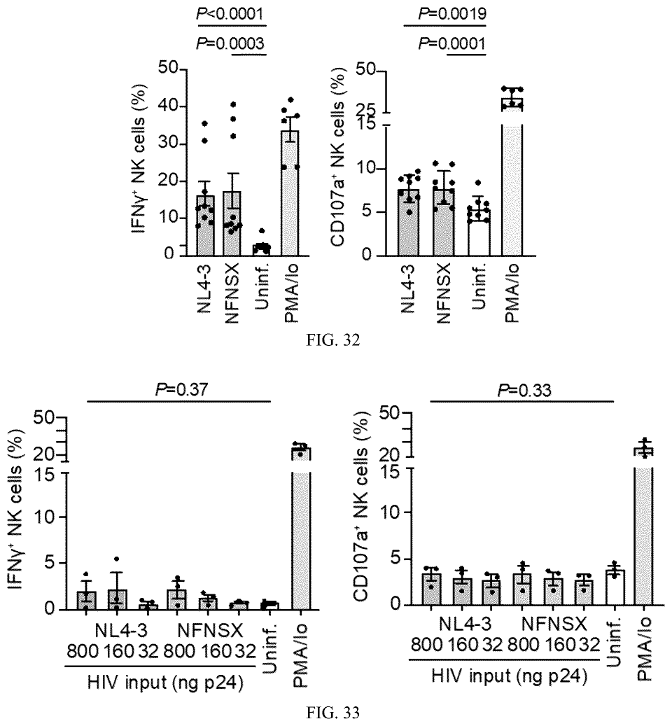

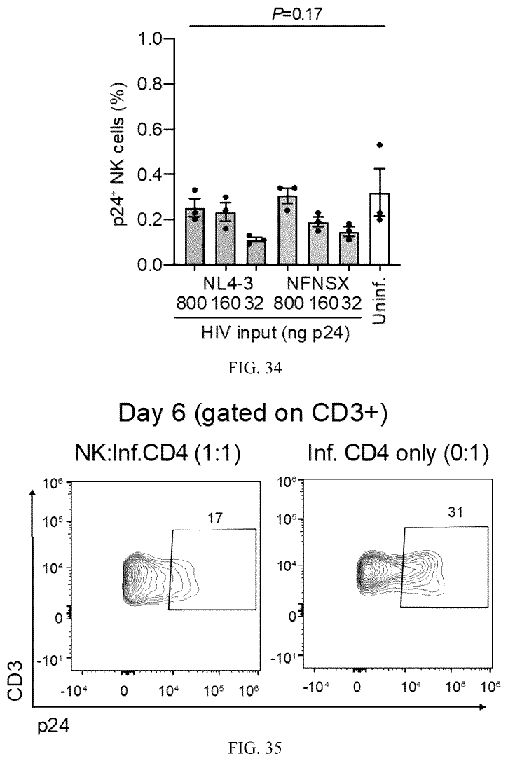

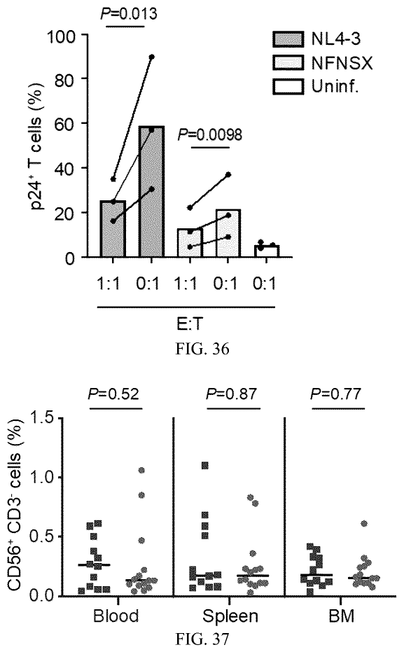

To assess the function of these NK cells, allogeneic CD4⁺ T cells were isolated from the PBMCs of healthy donors and performed CD3 and CD28 co-stimulation for 3 days prior to infection with 800 ng of p24 of X4-tropic NL4-3 or R5-tropic NFNSX for 24 h. NFNSX is derived from NL4-3 with the envelope cloned from the CCR-5 tropic JR-FL. The infected or uninfected CD4⁺ T cells were washed and cocultured with allogeneic NK cells at an effector-to-target ratio of 1:1 in media containing 20 ng per ml of recombinant human IL-2 (FIG. 31). Cocultures were analyzed 24 h and 48 h later to assess activation of NK cells and frequency of live infected T cells by flow cytometry. After 24 h of coculture the allogeneic NK cells exhibited higher levels of IFN-γ production and CD107a degranulation when cocultured with HIV-infected CD4⁺ T cells compared to allogeneic NK cells cocultured with uninfected CD4⁺ T cells or NK cells cultured alone (FIG. 32), indicating that allogeneic NK cells were specifically activated by HIV-infected CD4⁺ T cells. These results were consistent with previous findings that allogeneic NK cells are efficiently activated by HIV-infected CD4⁺ T cells. In addition, NK cells treated with varying doses of cell-free HIV supernatant in the absence of CD4⁺ T cells demonstrated no notable increase in levels of intracellular IFN-γ or degranulation (CD107a) after 24 h or intracellular p24 staining after 48 h (FIG. 33, FIG. 34), indicating that cell-free virus did not efficiently activate or replicate in NK cells. As expected, the frequency of infected p24⁺ CD4⁺ T cells was significantly lower among cocultures of allogeneic NK cells with infected CD4⁺ T cells compared to cultures containing infected CD4⁺ T cells without NK cells (FIG. 35, FIG. 36), indicating that allogeneic NK cells decreased HIV infection in CD4⁺ T cells.

### NK Cells Delay Viral Rebound of RS-Tropic HIV After ART Interruption

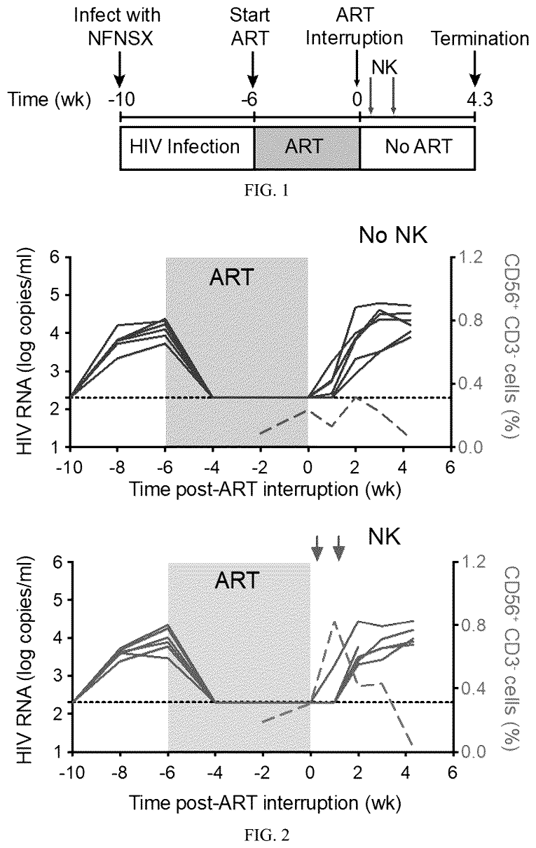

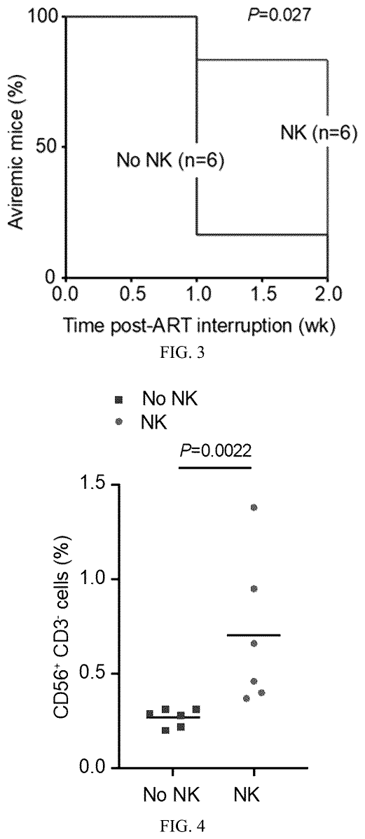

To study HIV latency in NSG-BLT mice, which achieve a high level of human immune cell reconstitution, NFNSX was intravenously injected and acute viremia was monitored by qRT-PCR in plasma samples for four weeks. Infected mice were administered ART comprised of raltegravir (RAL), emtricitabine (FTC), and tenofovir disoproxil fumarate (TDF) in the animal feed (FIG. 1). Once viremia was suppressed, ART was interrupted in the presence or absence of 5×10⁶ allogeneic human peripheral blood NK cells, which were administered at one- and six-days post-ART interruption. The donors for NK cell injections were randomly selected based on donor supply and not chosen based on KIR/HLA genotyping. Injections of NK cells in a mouse cohort were from the same human donor. Because there are no robust virologic or immunologic correlates to definitively identify all HIV reservoir cells, monitoring viral rebound after ART interruption was used as a primary endpoint in vivo. One week after ART interruption, rebound viremia occurred in five out of six (83%) mice in the control group and one out of six (17%) mice in the group receiving NK cells (FIG. 2). After two weeks of ART interruption, all mice in both groups rebounded. The group receiving NK cells exhibited a delay in viral rebound compared to the control group (P=0.027, FIG. 3). As expected, control mice displayed low frequencies of human CD56⁺CD3⁻ cells in the blood due to poor human NK cell development in BLT mice (FIG. 2, top). Compared to control mice, the mice receiving NK cells had 6-fold higher, but overall low levels (˜0.8%) of human CD56⁺CD3⁻ cells in the blood five days after the administration of NK cells (P=0.0022, FIG. 4), which then declined thereafter (FIG. 2, bottom). Indeed, adoptive transfer of allogeneic NK cells has limited engraftment. There were no notable differences between the frequencies of CD56⁺CD3⁻ cells in the blood, spleen, and bone marrow at necropsy between the two groups (FIG. 37). Although NK cells can downregulate CD56 expression and subpopulations of long-lived memory-like NK cells can exist, these results likely indicate the overall transient survival of adoptively transferred non-engineered NK cells.

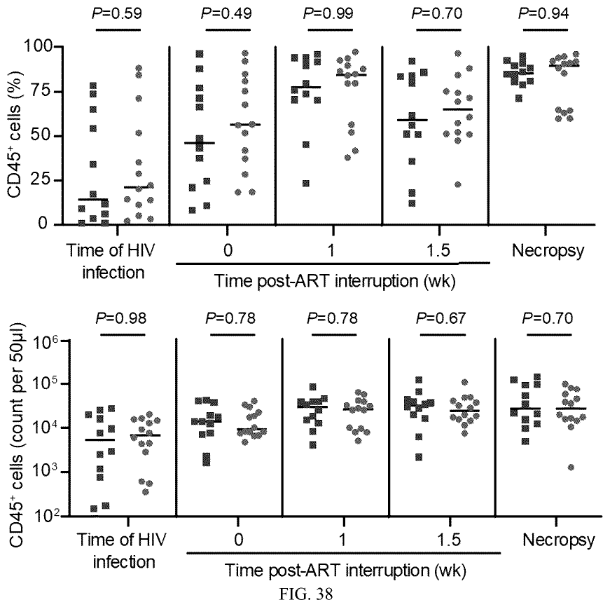

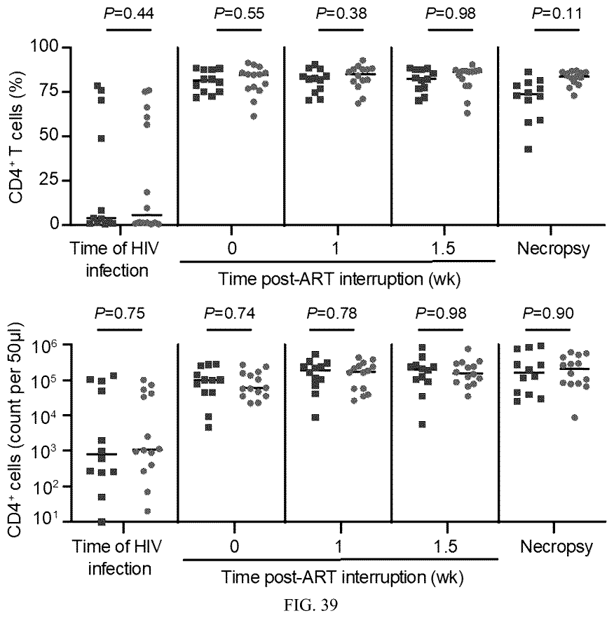

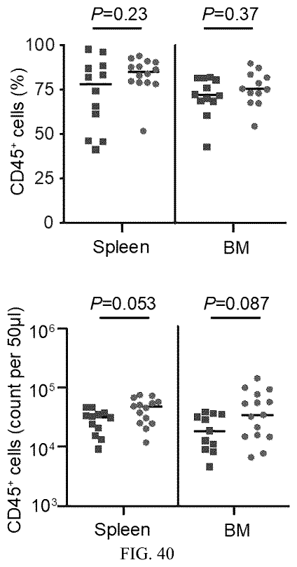

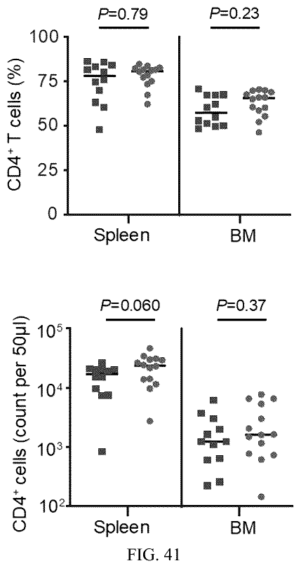

The levels of pre-ART viral infection and human immune cell engraftment likely did not account for differences in viral rebound because notable differences were not observed between the two groups (FIG. 5, FIG. 38 to FIG. 41). In addition, mice receiving NK cells did not demonstrate diminished frequencies or absolute counts of human CD45⁺ and CD4⁺ T cells compared to control mice, indicating allogeneic NK cells did not indiscriminately kill host immune cells, which is consistent with clinical studies demonstrating the overall safety of allogeneic NK cells in contrast to allogeneic T cells, which induce graft-versus-host disease.

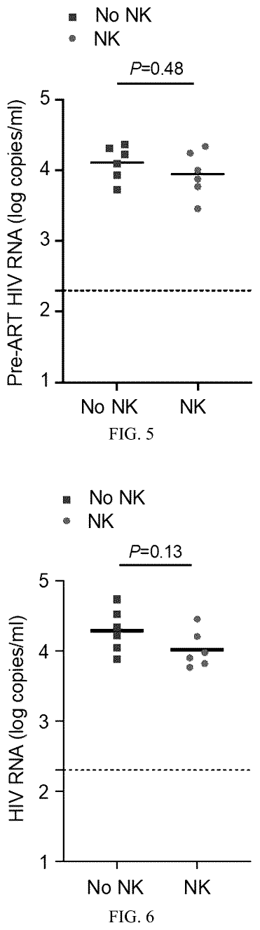

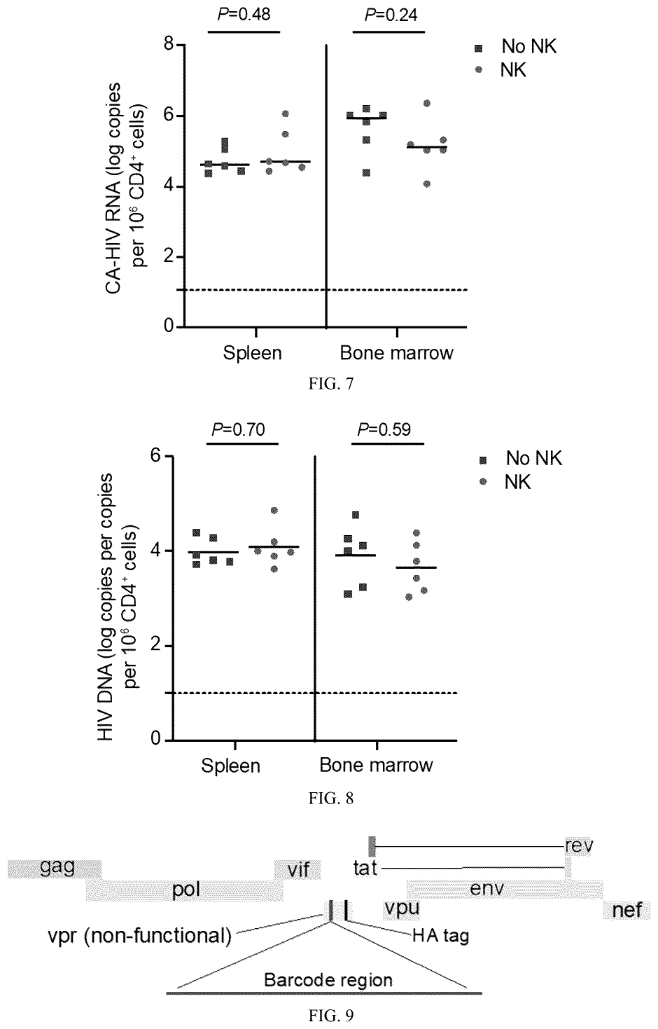

In addition, it was found that the level of viremia, cell-associated HIV RNA, and total HIV DNA of the spleens and bone marrow at necropsy were not significantly different between the mice treated with or without NK cells, which was expected given the rebounding virus was allowed to replicate and reseed new target cells during ART interruption (FIG. 6 to FIG. 8). These results indicate total HIV RNA and DNA measurements several weeks after ART interruption did not capture the dynamic effect that NK cells had on early viral rebound.

### NK Cells Also Delay Rebound of X4-Tropic Barcoded HIV

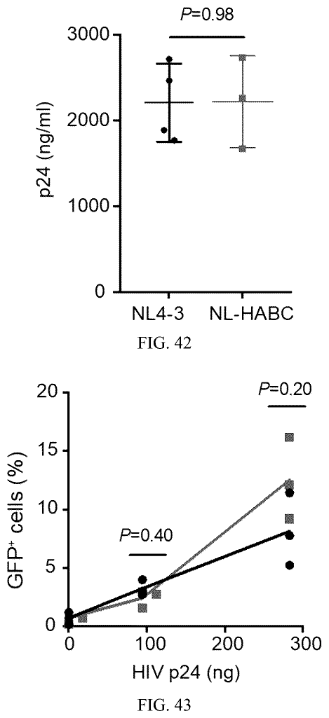

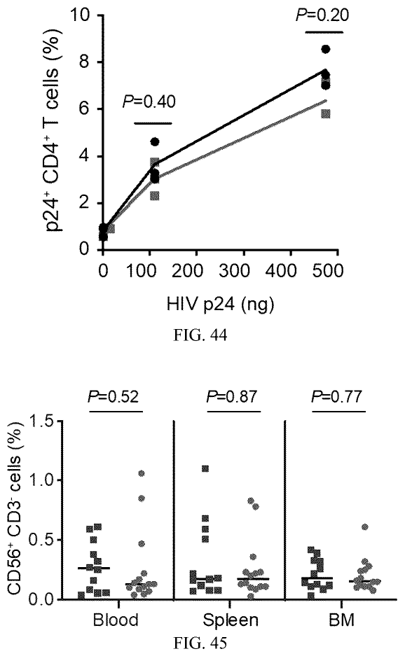

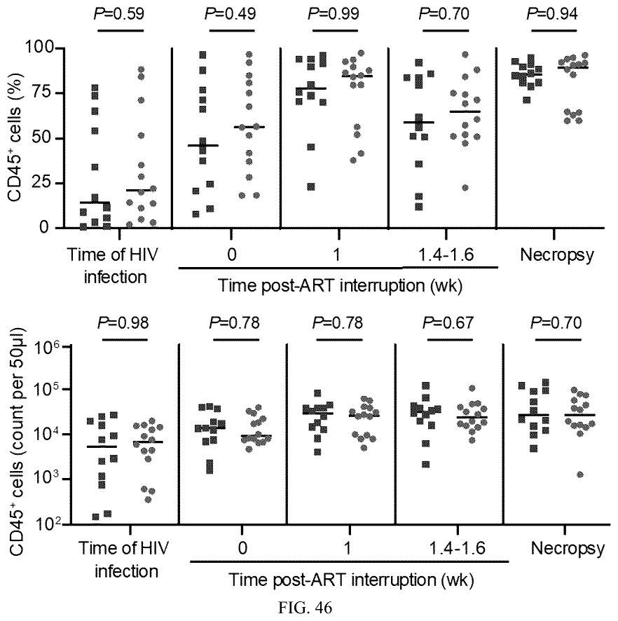

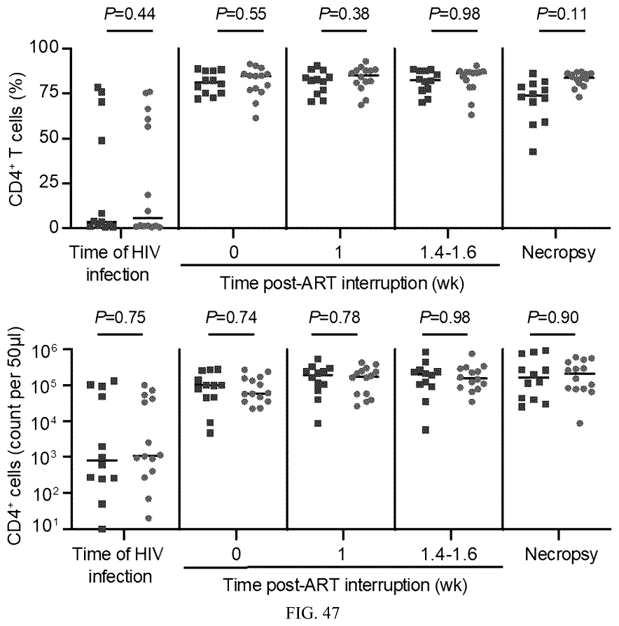

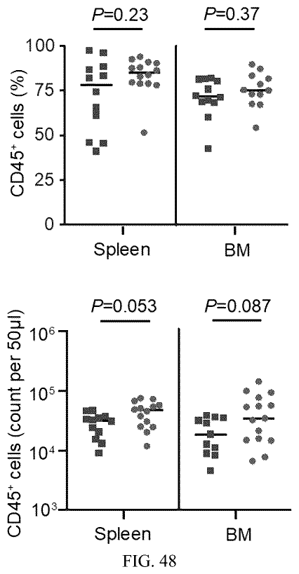

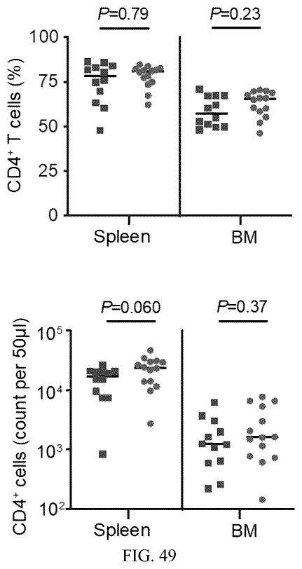

Whether HIV-1 barcoded technology can be used to quantify the effect that allogeneic human peripheral blood NK cells had on rebounding viral clones after ART interruption was investigated using a genetically barcoded HIV-1 containing a 21 bp genetic barcode inserted upstream of a hemagglutinin tag in the non-functional vpr region of the HIV strain NL-HA, which is an X4-tropic near full-length, replication-competent, pathogenic strain of NL4-3 (FIG. 9, FIG. 42 to FIG. 44). That is, allogeneic NK cells effectively delayed viral rebound of NL-HABC, which does not express vpr. Thus, multiple interactions involving inhibitory and activating receptors may likely mediate alloreactive NK response to infected target cells in vivo.

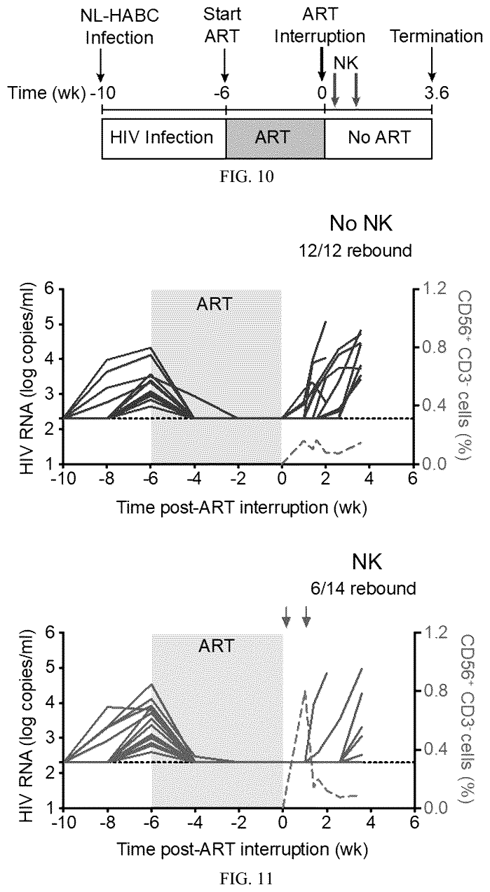

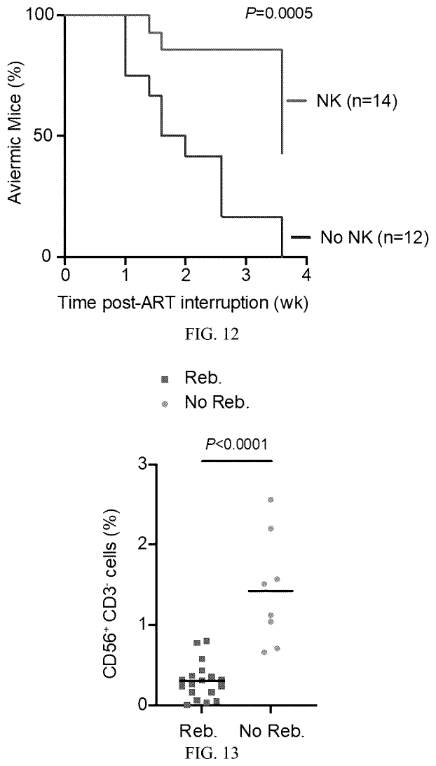

To validate that NK cells could also delay rebound of an X4-tropic strain, BLT mice were intravenously injected with NL-HABC and then four weeks post-infection initiated ART (RAL/FTC/TDF) for six weeks to reduce active viral replication (FIG. 10). Once viral loads were suppressed, ART was discontinued in the presence or absence of adoptively transferred allogeneic human peripheral blood NK cells similar to FIG. 1. Six out of 14 mice (43%) in the group receiving NK cells rebounded and showed a significant delay in viral rebound compared to the control group, in which all 12 mice (100%) rebounded (P=0.0005, FIG. 11 to FIG. 12). Again, the frequency of human CD56⁺CD3⁻ cells in the blood peaked and then declined in the group receiving NK cells (FIG. 11, bottom). Mice that did not rebound demonstrated a 4.8-fold higher frequency of human CD56⁺CD3⁻ cell in the blood compared to mice that did rebound (P<0.0001, FIG. 13), indicating that NK cells delay rebound.

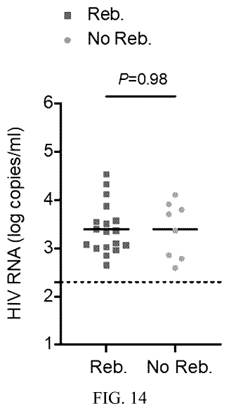

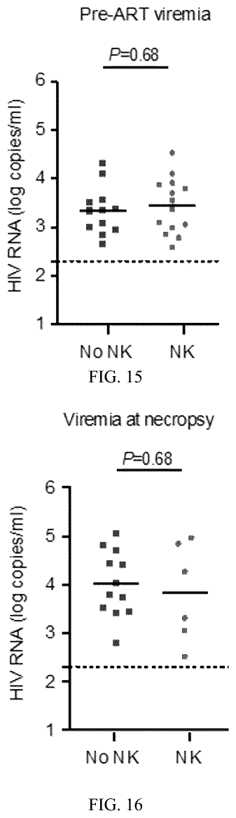

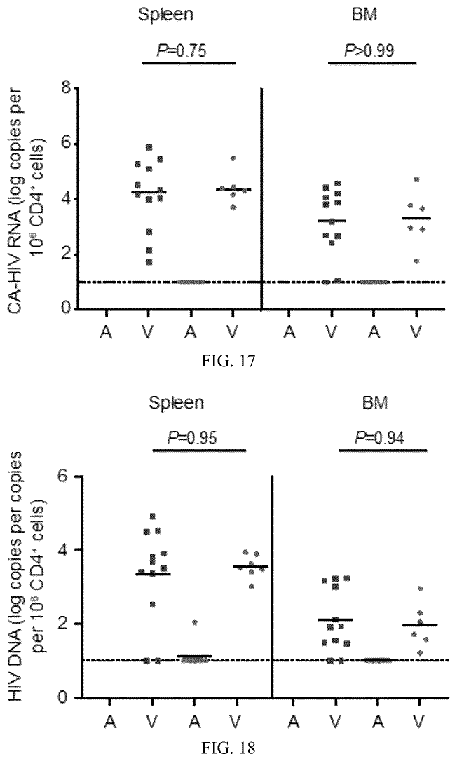

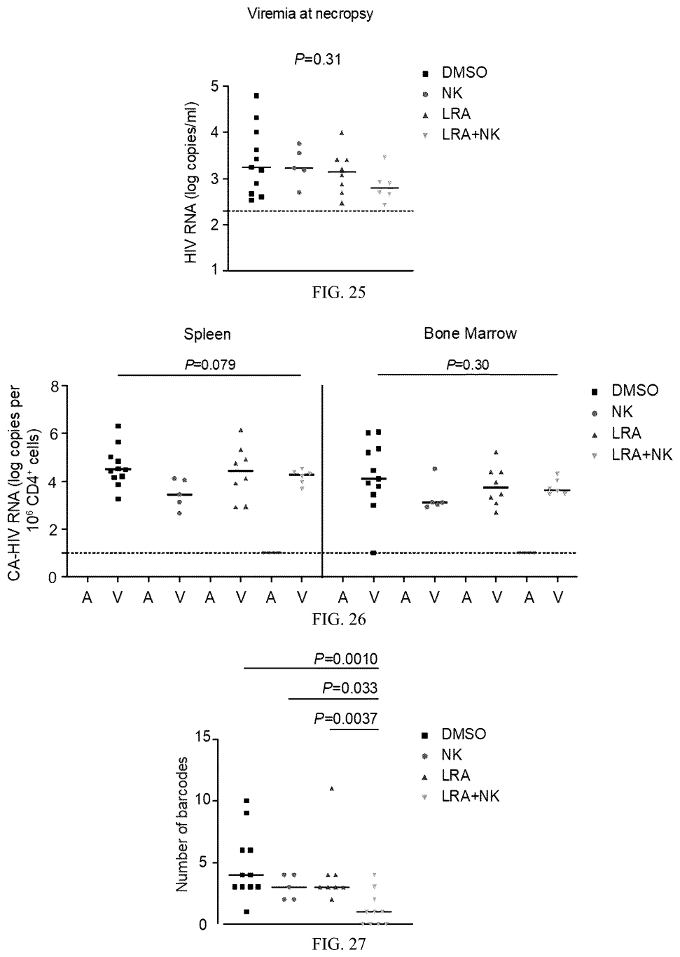

In addition, human immune cell engraftment between the mice that did or did not receive NK cells was not significantly different (FIG. 25 to FIG. 30). There was no significant difference between the pre-ART viral loads between mice that rebounded or did not rebound (FIG. 14) or mice that did or did not receive NK cells (FIG. 15). These results indicate pre-ART viral loads and the level of human immune cell engraftment likely did not contribute to differences in viral rebound. Among the mice that rebounded, there were no significant differences in the plasma viral loads at necropsy or level of cell-associated HIV RNA in the spleen and bone marrow between mice that did or did not receive NK cells (FIG. 16, FIG. 17), indicating again that once rebound occurred viral replication was robust in these compartments.

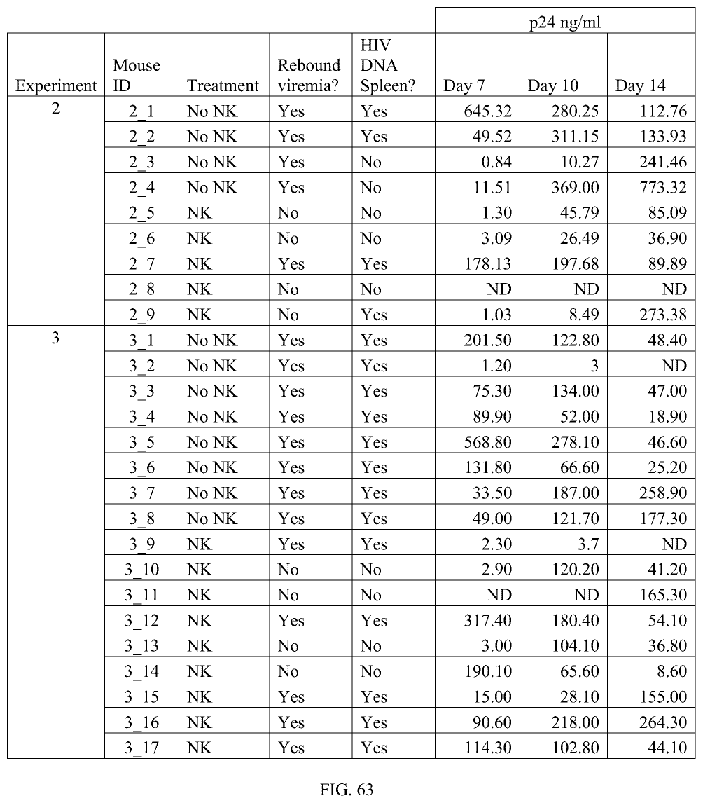

Among the mice with rebound viremia, there were no significant differences in the level of cell-associated HIV DNA in the spleen and bone marrow at necropsy between the control and treatment groups (FIG. 18), which was expected since rebound virus may reseed new target cells after ART interruption. Mice that did not display rebound viremia also had undetectable levels of cell-associated total HIV DNA in the spleens with the exception of one mouse in the NK group that had detectable HIV DNA in the spleen (mouse #2_17 in FIG. 63). In addition, although all the control mice demonstrated rebound plasma viremia, total HIV DNA from either the spleen in two out of twelve (17%) mice was unable to be detected by PCR using 200 ng of input cell-associated DNA (mouse #2_3 and #2_4 in FIG. 63), indicating possible sampling limitation since only a small portion of the total spleen was analyzed for HIV DNA viral loads.

NK Cells Reduce the Viral Growth after ART Interruption

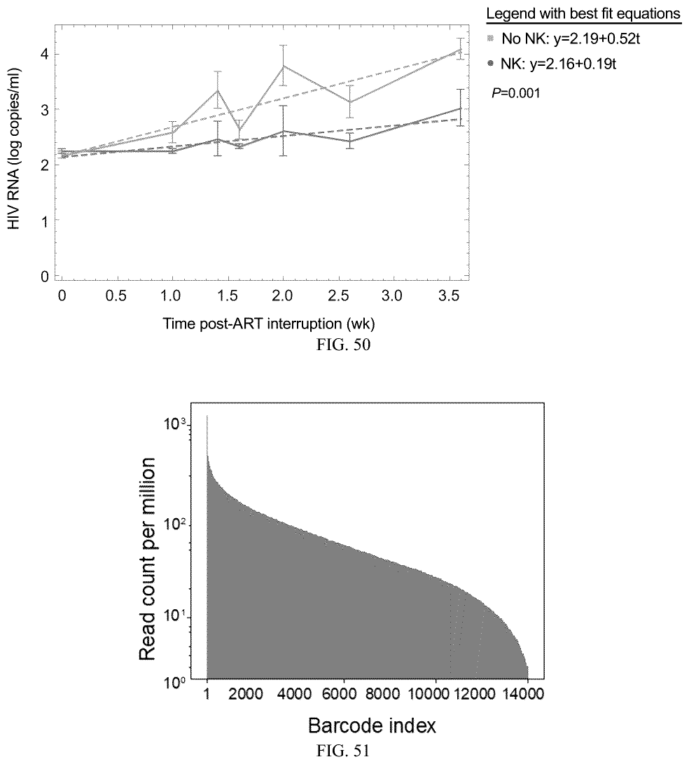

In addition to measuring time to rebound after ART interruption, viral growth kinetics between the mice that did or did not receive NK cells using the multiple timepoints in which viral loads were increasing prior to necropsy was compared (FIG. 50). The rate of virus spread following discontinuation of ART using a well-established mathematical model of virus dynamics was quantified. The exponential phase of virus growth during rebound viremia was focused on and this model was fit to the experimental data and found that the rate of increase of virus spread in the control group upon rebound was r=0.181 per day, while in the treatment group receiving NK cells it was r=0.087 per day, approximately a 2.1-fold decrease in the viral spread rate. These data indicate that NK cells target infected cells and thereby slowed the rate of productive infection of new uninfected target cells in vivo.

### NK Cells Reduce the Barcode Diversity of Rebounding Virus

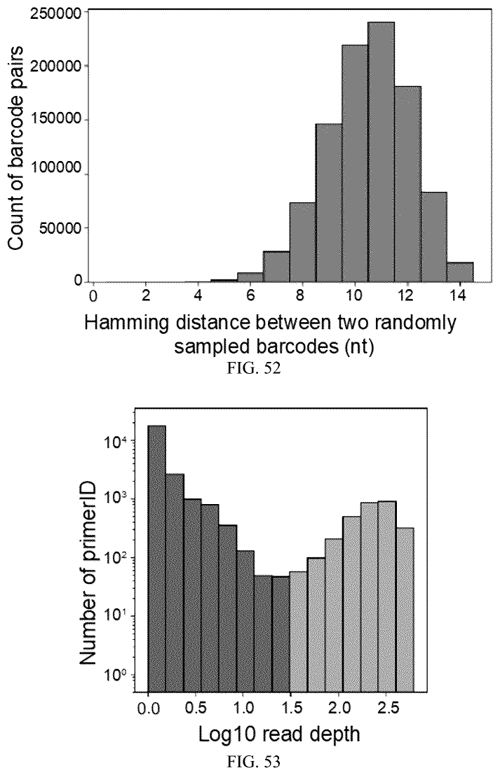

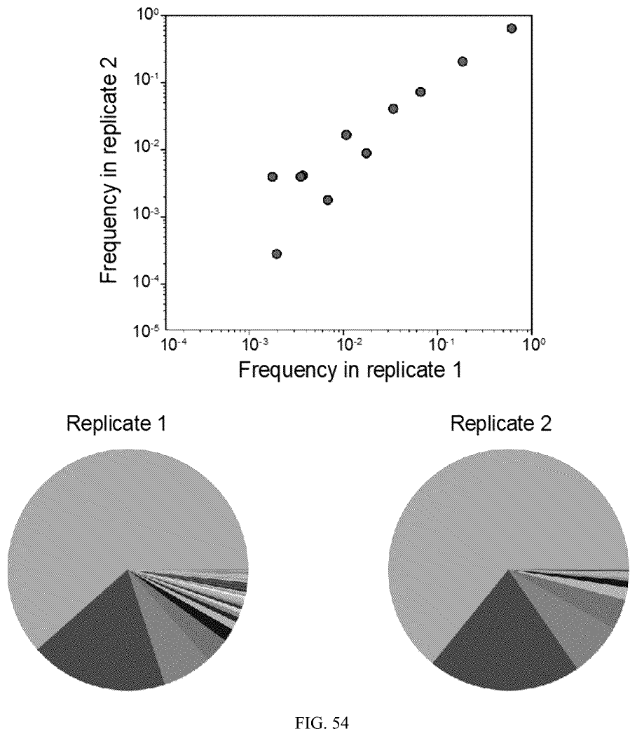

In addition to characterizing the size of the reservoir by measuring the time to rebound after ART interruption, HIV barcoded technology was used to measure the viral clone diversity of the reservoir. An error-reduction deep sequencing strategy was used to measure the number of HIV barcode variants that rebounded in the plasma, spleen, and bone marrow of each infected mouse after ART interruption. In this model, there were approximately 14,000 genetically different barcode clones in the virus preparation used to initially infect the animals (FIG. 51). Within the 21 bp barcode region, each barcode viral clone was different from one another on average by 10.5 bp, and the possibility that two barcodes were less than four bp different from each other was 0.04% (FIG. 52). Thus, barcode sequences with 4 or more bp differences from one another were considered unique clones. Although mutations in the barcode region may occur sporadically during viral replication, the chance of accruing at least 4 or more bp in the barcode region and thus misidentifying a mutated barcode as an independent clone was likely negligible. A primer ID tag on each RNA molecule during cDNA synthesis was employed to reduce sequencing errors and accurately quantify the starting number of RNA molecules (FIG. 53). The barcode number was not normalized to viral load because the occurrence of each barcode is not uniform since a few clones of barcode virus usually dominated the viral population. To assess the sensitivity and reproducibility of the barcode quantification, RNA molecules were sampled from the same population twice and found the frequencies of barcodes with 3-log difference were significantly correlated between the two replicates (r=0.92, P=6.7×10⁻⁵, FIG. 54).

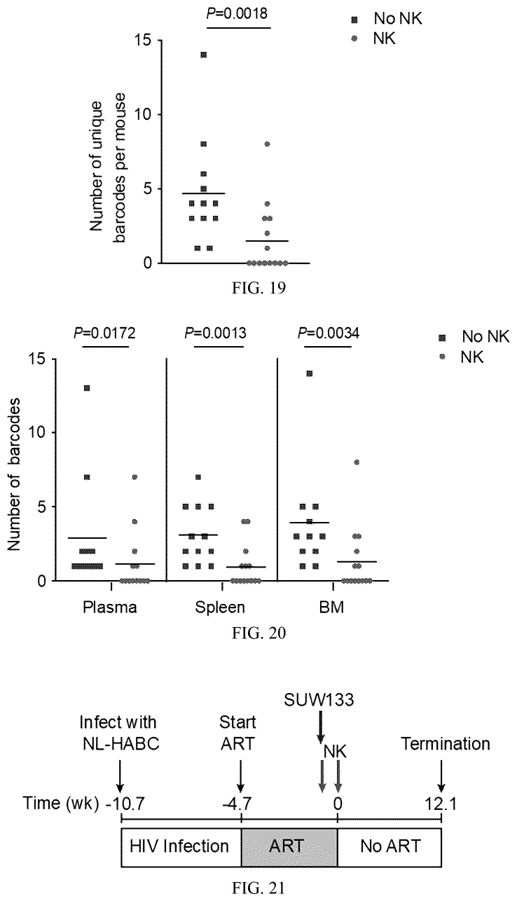

In contrast to total HIV RNA or DNA measurements, which are likely increased during ART interruption due to reseeding of the reservoir, the overall diversity of circulating clones was utilized as an additional measure to study the effect that NK cells had on the viral reservoir. The number of unique barcodes rebounding in each mouse was 3.2-fold higher in the control group compared to the group receiving NK cells (4.7 vs 1.5 mean unique barcodes, P=0.0018, FIG. 19). Mice receiving NK cells also had significantly lower numbers of rebound barcode variants in the plasma, spleen, and bone marrow. The major rebounding barcode variants were distributed throughout the plasma, spleen and bone marrow after ART discontinuation. Although 14,000 barcoded HIV clones were injected into humanized mice, on average only twenty to fifty barcodes were distributed in the mice prior to starting ART, and then after ART interruption only one to twenty barcodes were detected during viral rebound. Thus, there are likely in vivo bottlenecks that limit the number of barcode viral clones seeding the reservoir, and then further turnover of viral clones while animals are on ART. Overall, these results indicate NK cells eliminated some early rebounding viral clones, thereby decreasing the diversity of rebounding viral clones that were able to circulate during ART interruption.

Next, ex vivo co-stimulation of the splenocytes was performed with anti-CD3 and anti-CD28 antibodies, and then they were cocultured with CEM cells to propagate viral outgrowth for 14 days. Although total HIV DNA was not detected in the spleens and bone marrow of two control mice that had rebound viremia, replication-competent virus ex vivo was induced as measured by p24 ELISA from the splenocyte cocultures seeded with larger numbers of cells from these animals (mice #2_3 and #2_4 in FIG. 63). In addition, replication-competent virus was induced from the splenocytes of seven out of eight (88%) mice that received NK cells even though they lacked rebound plasma viremia (mice #2_5, #2_6, #2_8, #2_9, #3_10, #3_11, and #3_13, but not #3_14, induced virus in FIG. 63). These results indicate that although NK cells can efficiently eliminate reactivated cells and their viral clones as they rebound, they were not effective at kicking and killing latently infected cells out of latency.

Kick and Kill Employing SUW133 and NK Cells Delays, Inhibits, and/or Reduces Viral Rebound

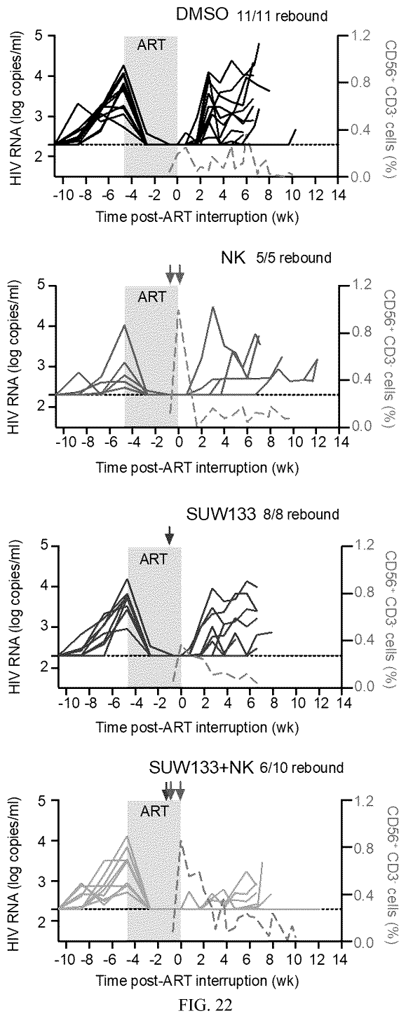

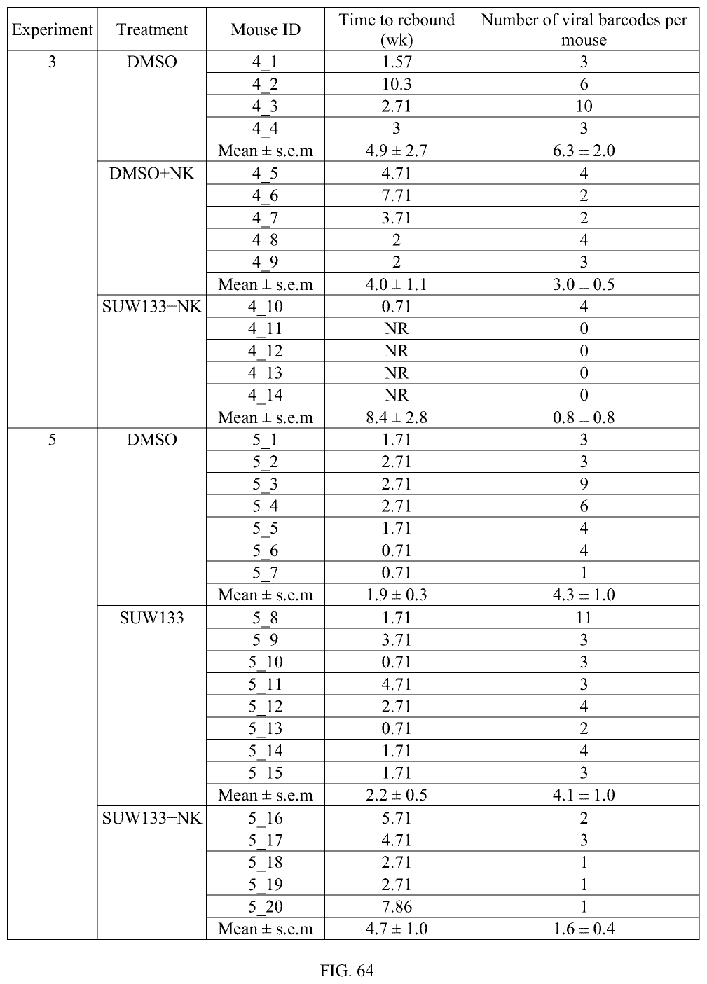

To determine whether combining a robust LRA with NK cells would effectively diminish the viral reservoir, as NK cells might eliminate cells induced to express virus due to the LRA activity, the following was performed. BLT mice were infected with NL-HABC, followed by ART to halt productive infection (FIG. 21, FIG. 64). Once mice were suppressed on ART, a subset of mice received a single intraperitoneal injection of 2 μg of SUW133 per mouse to reverse latency, followed by an intravenous injection of 5×10⁶ allogeneic human NK cells eight hours later. Reactivation and killing of activated infected cells was allowed to occur while the mice were maintained on ART. ART was then discontinued, and mice were concurrently administered one more intravenous injection of 5×10⁶ NK cells. A subset of control mice received SUW133, but no NK cell injections. All remaining control mice received DMSO as a vehicle control instead of SUW133. A subset of the mice receiving DMSO also received NK cell injections. Plasma viral loads were monitored after ART interruption for 12.1 weeks (FIG. 22). In these experiments, TKO-BLT mice, which can be robustly infected with HIV and are less susceptible to GVHD compared to NSG-BLT, were used and allowed monitoring the mice after ART interruption for a longer interval.

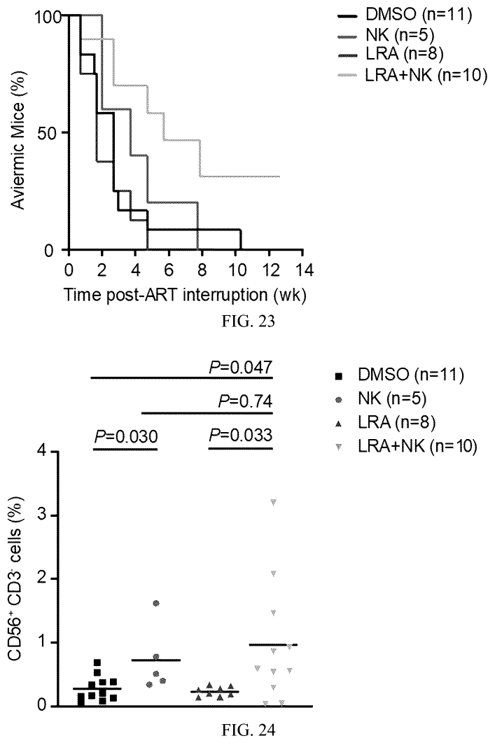

Four out of ten (40%) mice receiving the combination of SUW133 plus NK cells displayed no rebound viremia despite monitoring animals for an extended time after ART interruption. In contrast, all control mice receiving SUW133 alone, NK cells alone, or DMSO alone rebounded during the lengthened interval of ART interruption (FIG. 22). Mice receiving SUW133 plus NK cells demonstrated a significant or trend towards delay to rebound after ART interruption in comparison to the other groups (P=0.012, SUW133+NK vs DMSO; P=0.0037, SUW133 plus NK vs SUW133; P=0.09, SUW133 plus NK vs NK; log-rank Mantel-Cox test; FIG. 23). The mice receiving SUW133 alone or NK cells alone did not demonstrate a significant delay in rebound compared to mice receiving DMSO alone (P=0.59, SUW133 vs DMSO; P=0.47, NK vs DMSO, log-rank Mantel-Cox test, FIG. 23).

In contrast to mice that received NK cells in FIG. 9 to FIG. 14, in this experiment all the mice receiving NK cells alone eventually rebounded and no significant delay to viral rebound was observed compared to control mice receiving DMSO alone (FIG. 23). To note, the mice receiving NK cells alone in this experiment initially appeared to have a delay in rebound compared to mice receiving DMSO alone during the first two to four weeks post-ART interruption, but as viral loads were monitored for a longer period after ART was discontinued this trend did not reach statistical significance. In this experiment mice were administered NK cells while they were on ART, thus the level of peripheral blood CD56⁺CD3⁻ cells peaked while animals were on ART (FIG. 22), indicating that HIV-expressing cells may not have been present for NK cells to target in the absence of an LRA.

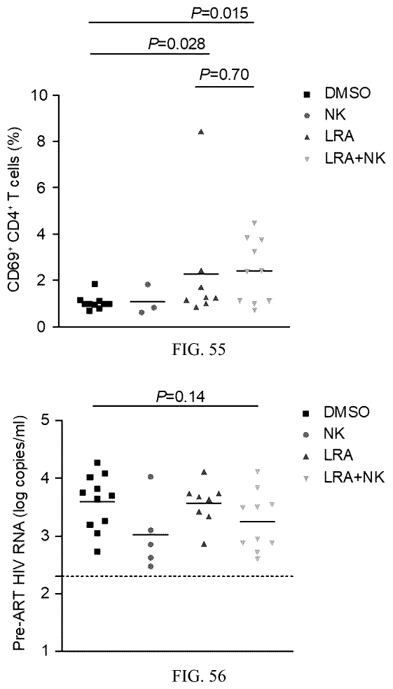

To assess whether the SUW133 was activating latent cells in vivo, the expression of the activation marker CD69 on CD4⁺ T cells in vivo was measured and CD69 was found to be significantly elevated on peripheral CD4⁺ T cells in the blood five days after administration of SUW133 among mice that received either SUW133 plus NK cells or SUW133 alone (FIG. 55). There was no significant difference in CD69 expression on CD4⁺ T cells between mice that received SUW133 alone or SUW133 plus NK cells. These results are consistent with previous report that CD69 is maximally expressed on CD4⁺ T cells in vivo approximately one to two days post-SUW133 injection, and then declined, but can be detected for up to nine days post-injection. In addition, among the mice that received NK cells alone or in combination with SUW133, the level of CD56⁺CD3⁻ cells in the blood increased over the five days post-NK cell injection as expected (FIG. 22, FIG. 24). Among the mice receiving SUW133 plus NK cells, the engraftment period of NK cells likely coincided with the period of latency reversal induced by SUW133, thereby allowing NK cell killing of latent cells upregulating activation or stress markers. Altogether, these results indicate that the combination of SUW133 and NK cells provided the most durable delay in rebound over these longer timescales compared with either individual treatment alone.

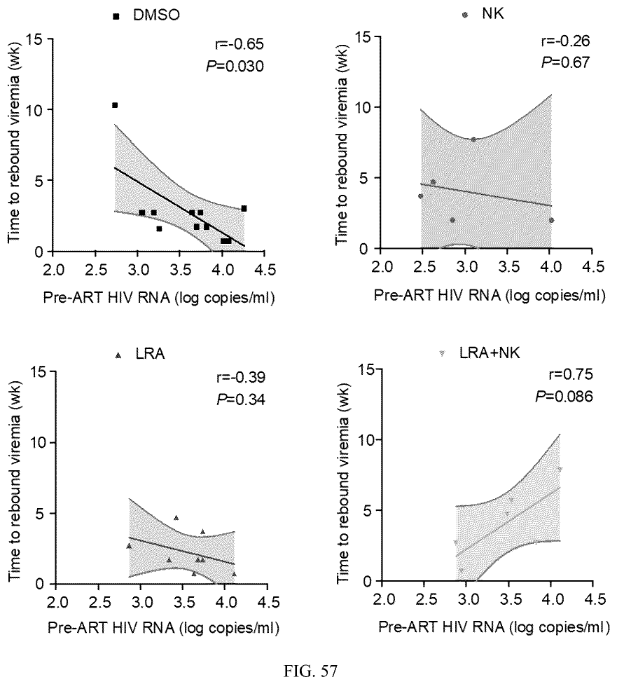

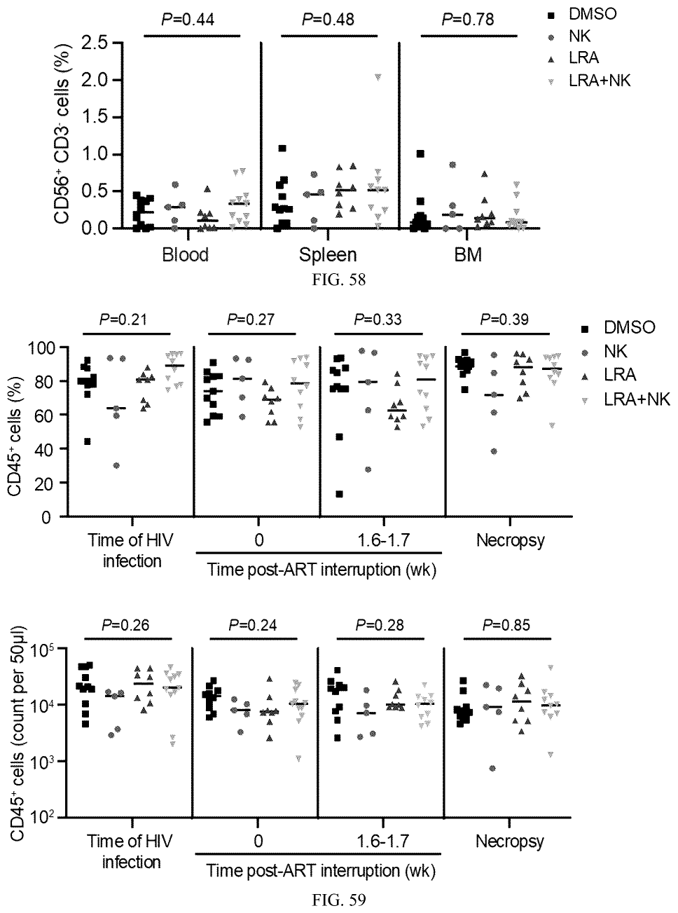

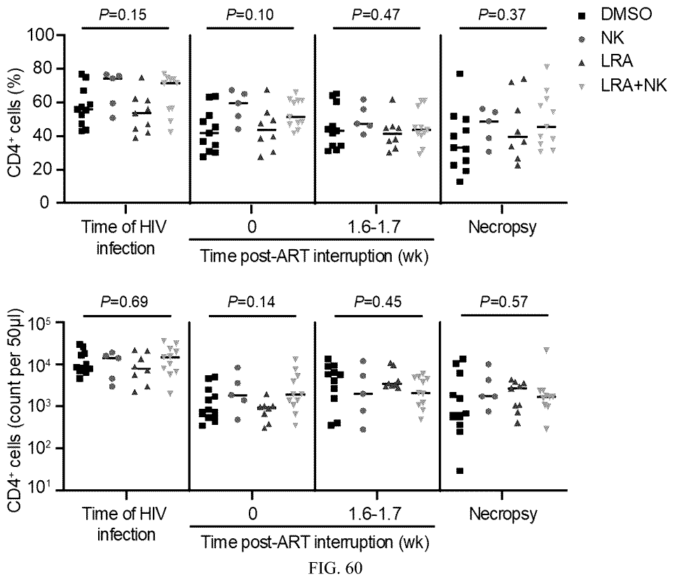

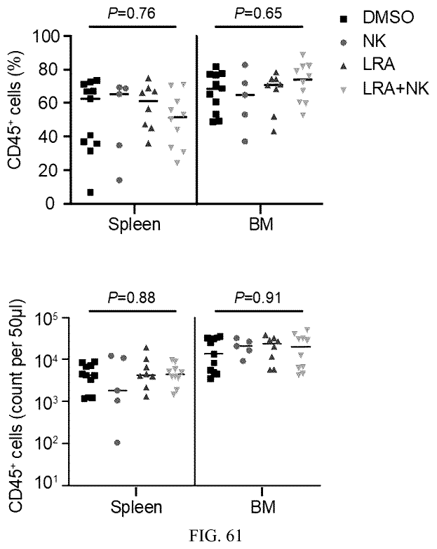

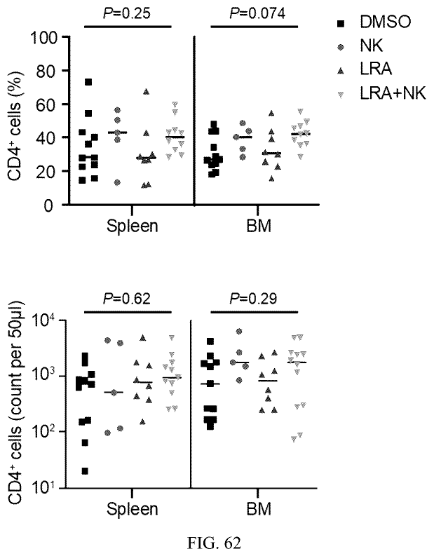

There was no significant difference between the pre-ART viral loads among the groups of mice (FIG. 56). Next, whether there was a correlation between time to rebound and pre-ART viral loads among the groups of mice was assessed. Pre-ART viral loads was inversely correlated with time to rebound in mice that received DMSO only (r=−0.65; P=0.030, Pearson correlation test, FIG. 57), indicating the extent of viral infection before ART initiation may play a role in the timing of viral rebound after ART interruption. Similar trends were seen in mice that received SUW133 alone or NK cells alone, but not LRA plus NK cells. These results indicate that the effectiveness of the kick and kill approach may not significantly correlate to the extent of the initial seeding of the reservoir. In addition, the frequencies and absolute counts of human immune cells between the different groups of mice were not significantly different (FIG. 58 to FIG. 62), indicating levels of human immune cell engraftment likely did not contribute to differences in viral rebound between treatment groups.

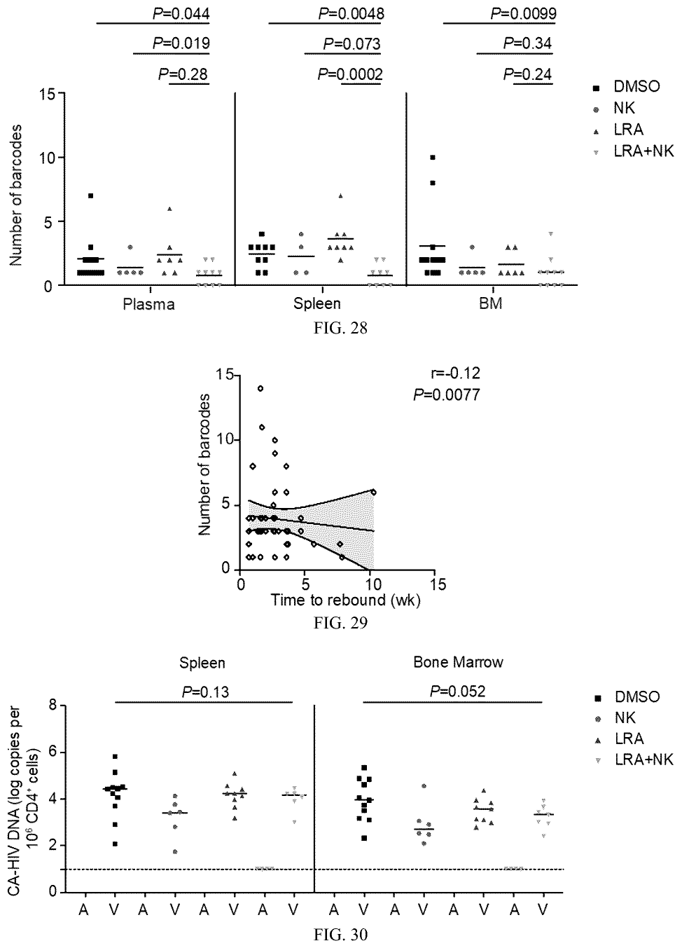

Next, plasma viremia and cell-associated HIV RNA in the tissues were analyzed. Among the mice with rebound viremia, the viral loads at necropsy were not significantly different between treatment and control groups (FIG. 25, FIG. 26), which was expected since viral replication was allowed to proceed in rebounding animals. The viral RNA was deep sequenced to analyze the barcode diversity of the rebounding virus in each mouse. Mice receiving the combination of SUW133 plus NK cells had 1.1 mean unique barcodes per mouse, which was significantly lower than 4.7, 3.0, and 4.1 mean unique barcodes from mice receiving DMSO alone, NK cells alone, and SUW133 alone, respectively (FIG. 27). There were no notable differences in the numbers of unique barcodes between the groups of mice receiving NK cells alone, SUW133 alone, and DMSO alone (P=0.25, NK vs DMSO; P=0.52, SUW133 vs DMSO; P=0.65, NK vs SUW133; two-tailed Mann-Whitney test; FIG. 27). A similar trend was also observed when comparing barcode diversities in the plasma, spleen, and bone marrow of these groups (FIG. 28), indicating the combination of SUW133 plus NK cells more effectively diminished the diversity of rebounding viral clones compared to either SUW133 alone, NK cells alone, or the DMSO vehicle control. In addition, a significant inverse correlation between time to rebound and the number of unique barcodes was found among all the rebounding animals in this study (FIG. 29), indicating that the breadth of barcode diversity significantly correlates with the time to rebound after ART interruption. Thus, nucleic acid barcoding may be used to characterize HIV reservoirs.

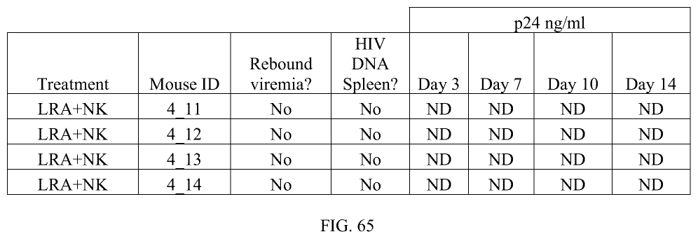

Among the four mice that received the combination of SUW133 plus NK cells and did not display rebound viremia, cell-associated HIV DNA was not detected in the tissues (FIG. 30). In comparison, cell-associated HIV DNA was detectable from the spleen and bone marrow of all rebounding animals. Enough splenocytes were obtained from the four mice, which received SUW133 plus NK cells and lacked rebound viremia, to perform an ex vivo co-stimulation of the splenocytes. Replication-competent virus from the splenocyte cocultures by p24 ELISA was unable to be detected (FIG. 65). These results indicate that the administration of SUW133 plus NK cells efficiently eliminated cells harboring replication-competent virus in the spleens in a subset of mice.

### Treatment Methods

Contemplated herein are methods for treating HIV in subjects and reducing the amount of HIV in subjects, e.g., by reducing or eliminating reservoirs of latent HIV in subjects. The methods comprise co-administering to the subjects (a) one or more LRAs such as a bryostatin compound, e.g. SUW133, and (b) NK cells. The one or more LRAs, such as a bryostatin compound and the NK cells may be administered together as a single pharmaceutical composition or as separate pharmaceutical compositions.

As used herein, “co-administration” refers to the administration of at least two different agents, i.e., a first agent (e.g., one or more LRAs, such as a bryostatin compound) and a second agent (e.g., NK cells) to a subject. In some embodiments, the co-administration is concurrent. In embodiments involving concurrent co-administration, the agents may be administered as a single composition, e.g., an admixture, or as two separate compositions. In some embodiments, the first agent is administered before and/or after the administration of the second agent. Where the co-administration is sequential, the administration of the first and second agents may be separated by a period of time, e.g., minutes, hours, or days. Those of skill in the art understand that the formulations and/or routes of administration of the various agents or therapies used may vary. The appropriate dosage for co-administration can be readily determined by one skilled in the art. In some embodiments, when two or more agents are co-administered, the respective agents are administered at lower dosages than appropriate for their administration alone.

In some embodiments, the one or more LRAs, such as a bryostatin compound (e.g., SUW133) and the NK cells are administered concurrently. In some embodiments, the one or more LRAs and the NK cells are administered after cessation of ART. In some embodiments, the one or more LRAs is administered about 2-24 days after ART cessation. In some embodiments, the one or more LRAs is administered 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, or 24 days after ART cessation. In some embodiments, the NK cells are administered after the administration of one or more LRAs. In some embodiments, the NK cells are administered about 2-14 hours, about 4-12 hours, about 6-10 hours, or about 8 hours after administration of the one or more LRAs. In some embodiments, the one or more LRAs is administered 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, or 24 days after ART cessation, and the NK cells are administered about 2-14 hours, about 4-12 hours, about 6-10 hours, or about 8 hours after administration of the one or more LRAs.

In some embodiments, the one or more LRAs and/or the NK cells are administered in therapeutically effective amounts. As used herein, a “therapeutically effective amount” refers to an amount that may be used to provide a therapeutic result in a subject as compared to a control, such as a placebo. In the case of LRAs such as bryostatin compounds, a therapeutically effective amount is an amount sufficient to reactivate HIV from latency in a subject. In the case of NK cells, a therapeutically effective amount is an amount sufficient to reduce the amount of HIV infected cells in a subject. A skilled artisan will appreciate that certain factors may influence the amount required to effectively treat a subject, including the degree of the condition or symptom to be treated, previous treatments, the general health and age of the subject, and the like.

In some embodiments, a therapeutically effective amount of an LRA, such as a bryostatin compound, is about 25-75, about 35-65, about 40-50, or about 45 mg/m² surface area of the subject. In some embodiments, a therapeutically effective amount of an LRA, such as a bryostatin compound, is about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, or about 120 mg.

In some embodiments, a therapeutically effective amount of a NK cells is about 1-30×10⁶, about 10-30×10⁶, or about 25×10⁶ NK cells/kg body weight of the subject. In some embodiments, a therapeutically effective amount of an LRA, such as a bryostatin compound, is about 0.75-2, about 1.0-1.75, about 1.1-1.3, about 1.25 mg/kg body weight of the subject. In some embodiments, the therapeutically effective amount of the NK cells is about 60×10⁶, about 90×10⁶, about 120×10⁶, about 150×10⁶, about 180×10⁶, about 210×10⁶, about 240×10⁶, about 270×10⁶, about 300×10⁶, about 330×10⁶, about 360×10⁶, about 390×10⁶, about 420×10⁶, about 450×10⁶, about 480×10⁶, about 510×10⁶, about 540×10⁶, about 570×10⁶, about 600×10⁶, about 630×10⁶, about 660×10⁶, about 690×10⁶, about 720×10⁶, about 750×10⁶, about 780×10⁶, about 810×10⁶, about 840×10⁶, about 870×10⁶, about 900×10⁶, about 930×10⁶, about 960×10⁶, about 990×10⁶, about 1020×10⁶, about 1050×10⁶, about 1080×10⁶, about 1110×10⁶, about 1140×10⁶, about 1170×10⁶, about 1200×10⁶, about 1230×10⁶, about 1260×10⁶, about 1290×10⁶, about 1320×10⁶, about 1350×10⁶, about 1380×10⁶, about 1410×10⁶, about 1440×10⁶, about 1470×10⁶, about 1500×10⁶, about 1530×10⁶, about 1560×10⁶, about 1590×10⁶, about 1620×10⁶, about 1650×10⁶, about 1680×10⁶, about 1710×10⁶, about 1740×10⁶, about 1770×10⁶, or about 1800×10⁶.

In some embodiments, the LRA, such as a bryostatin compound, is administered intraperitoneally. In some embodiments, the NK cells are administered intravenously. In some embodiments, the NK cells are administered about 4-12 hours, about 6-10 hours, or about 8 hours after the administration of the LRA. In some embodiments, ART is continued for about 3-7 days, about 4-6 days, or about 5 days after administration of the LRA and NK cells. In some embodiments after ART is interrupted, the subject is administered a second therapeutically effective amount of a NK cells. In some embodiments, the LRA, such as a bryostatin compound, is administered intraperitoneally and the NK cells are administered intravenously about 4-12 hours, about 6-10 hours, or about 8 hours after the administration of the LRA; ART is continued for about 3-7 days, about 4-6 days, or about 5 days after administration of the LRA and NK cells, and then ART is interrupted and the subject is administered a second therapeutically effective amount of a NK cells.

In some embodiments, the methods further comprise administering one or more supplementary agents. Examples of suitable supplementary agents include cytokines such as IL-2, IL-12, IL-15, IL-18, and CCL5; curcumin; ginseng extract; garlic extract; resveratrol; kumquat pericarp extract; prostratin, lectins; polysaccharides; and the like.

### Compositions, Kits, and Combination Products

Contemplated herein are compositions (including pharmaceutical compositions), kits, and combination products comprising (a) one or more LRAs such as a bryostatin compound, e.g. SUW133, in combination with (b) NK cells. As used herein, term “pharmaceutical composition” refers to a composition suitable for pharmaceutical use in a subject. A pharmaceutical composition generally comprises a therapeutically effective amount of an active agent (e.g., an LRA and/or NK cells) and a pharmaceutically acceptable carrier. Preferred pharmaceutical compositions comprise a therapeutically effective amount of the given active agent and a pharmaceutically acceptable vehicle. In some embodiments, the compositions, kits, and combination products additionally include one or more supplementary agents. Examples of suitable supplementary agents include cytokines such as IL-2, IL-12, IL-15, IL-18, and CCL5; curcumin; ginseng extract; garlic extract; resveratrol; kumquat pericarp extract; prostratin, lectins; polysaccharides; and the like. In some embodiments, the kits and combination products additionally contain one or more reagents or drug delivery devices, e.g., a pre-filled syringe.

In some embodiments, the kits and combination products include a carrier, package, or container that may be compartmentalized to receive one or more containers, such as vials, tubes, and the like. In some embodiments, the kits optionally include an identifying description or label or instructions relating to its use. In some embodiments, the kits include information prescribed by a governmental agency that regulates the manufacture, use, or sale of compounds and compositions as contemplated herein.

In some embodiments, the compositions, kits, and combination products comprise the one or more LRAs and/or the NK cells in therapeutically effective amounts. In some embodiments, a therapeutically effective amount of an LRA, such as a bryostatin compound, is about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, or about 120 mg.

In some embodiments, the therapeutically effective amount of the NK cells is about 60×10⁶, about 90×10⁶, about 120×10⁶, about 150×10⁶, about 180×10⁶, about 210×10⁶, about 240×10⁶, about 270×10⁶, about 300×10⁶, about 330×10⁶, about 360×10⁶, about 390×10⁶, about 420×10⁶, about 450×10⁶, about 480×10⁶, about 510×10⁶, about 540×10⁶, about 570×10⁶, about 600×10⁶, about 630×10⁶, about 660×10⁶, about 690×10⁶, about 720×10⁶, about 750×10⁶, about 780×10⁶, about 810×10⁶, about 840×10⁶, about 870×10⁶, about 900×10⁶, about 930×10⁶, about 960×10⁶, about 990×10⁶, about 1020×10⁶, about 1050×10⁶, about 1080×10⁶, about 1110×10⁶, about 1140×10⁶, about 1170×10⁶, about 1200×10⁶, about 1230×10⁶, about 1260×10⁶, about 1290×10⁶, about 1320×10⁶, about 1350×10⁶, about 1380×10⁶, about 1410×10⁶, about 1440×10⁶, about 1470×10⁶, about 1500×10⁶, about 1530×10⁶, about 1560×10⁶, about 1590×10⁶, about 1620×10⁶, about 1650×10⁶, about 1680×10⁶, about 1710×10⁶, about 1740×10⁶, about 1770×10⁶, or about 1800×10⁶.

It should be noted that a therapeutically effective amount may be administered as a single dose or as a series of several doses. The dosages used for treatment may increase or decrease over the course of a given treatment. Optimal dosages for a given set of conditions may be ascertained by those skilled in the art using dosage-determination tests and/or diagnostic assays in the art. Dosage-determination tests and/or diagnostic assays may be used to monitor and adjust dosages during the course of treatment.

Pharmaceutical compositions may be formulated for the intended route of delivery and administered to subjects accordingly using methods in the art. Pharmaceutical compositions may include one or more of the following: a pharmaceutically acceptable vehicle, pH buffered solutions, adjuvants (e.g., preservatives, wetting agents, emulsifying agents, and dispersing agents), liposomal formulations, nanoparticles, dispersions, suspensions, or emulsions, as well as sterile powders for reconstitution into sterile injectable solutions or dispersions. The compositions and formulations may be optimized for increased stability and efficacy using methods in the art. See, e.g., Carra et al., (2007) Vaccine 25:4149-4158.

As used herein, a “pharmaceutically acceptable vehicle” or “pharmaceutically acceptable carrier” are used interchangeably and refer to solvents, dispersion media, coatings, antibacterial and antifungal agents, isotonic and absorption delaying agents, and the like, that are compatible with pharmaceutical administration and comply with the applicable standards and regulations, e.g., the pharmacopeial standards set forth in the United States Pharmacopeia and the National Formulary (USP-NF) book, for pharmaceutical administration. Thus, for example, unsterile water is excluded as a pharmaceutically acceptable carrier for, at least, intravenous administration. Pharmaceutically acceptable vehicles include those known in the art. See, e.g., Remington: The Science and Practice of Pharmacy 20th ed (2000) Lippincott Williams & Wilkins, Baltimore, MD.

The pharmaceutical compositions may be provided in dosage unit forms. As used herein, a “dosage unit form” refers to physically discrete units suited as unitary dosages for the subject to be treated; each unit containing a predetermined quantity of the one or more active ingredient(s) calculated to produce the desired therapeutic effect in association with the required pharmaceutically acceptable carrier. The specification for the dosage unit forms of the invention are dictated by and directly dependent on the unique characteristics of the given active ingredient(s) and desired therapeutic effect to be achieved, and the limitations inherent in the art of compounding such an active compound for the treatment of individuals.

Toxicity and therapeutic efficacy of the compositions and methods according to the instant invention and compositions thereof can be determined using cell cultures and/or experimental animals and pharmaceutical procedures in the art. For example, one may determine the lethal dose, LC₅₀ (the dose expressed as concentration x exposure time that is lethal to 50% of the population) or the LD₅₀ (the dose lethal to 50% of the population), and the ED₅₀ (the dose therapeutically effective in 50% of the population) by methods in the art. The dose ratio between toxic and therapeutic effects is the therapeutic index and it can be expressed as the ratio LD₅₀/ED₅₀. Compositions and methods which exhibit large therapeutic indices are preferred. While compositions and methods that result in toxic side-effects may be used, care should be taken to design a delivery system that targets such compounds to the site of treatment to minimize potential damage to uninfected cells and, thereby, reduce side-effects.

The data obtained from the cell culture assays and animal studies can be used in formulating a range of dosages for use in humans. Preferred dosages provide a range of circulating concentrations that include the ED₅₀ with little or no toxicity. The dosage may vary depending upon the dosage form employed and the route of administration utilized. Therapeutically effective amounts and dosages of active ingredient(s) can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. Additionally, a dosage suitable for a given subject can be determined by an attending physician or qualified medical practitioner, based on various clinical factors.

The following examples are intended to illustrate but not to limit the invention.

### EXAMPLES

Study design: No statistical methods were used to predetermine sample size. The investigators were not blinded to allocation during experiments, so they could assess whether the treatments were being tolerated by the animals. However, initial data and sequence analysis was conducted in a blinded fashion with respect to treatment.

Cells and cell lines: De-identified PBMCs from healthy human donors were obtained under informed consent from the UCLA AIDS Institute Virology Core Laboratory under IRB approval then provided to investigators in an anonymized fashion. NK cells were isolated using CD56 MicroBeads (Miltenyi) with 75-85% purity. Cells were stained with CD158(KIR)-FITC (clone HP-MA4), CD244(2B4)-PE (clone C1.7), NKp80-APC (clone 5D12), CD336(NKp44)-PerCp/Cy5.5 (clone P44-8), CD314(NKG2D)-PE-Dazzle594 (clone 1D11), CD159a(NKG2A)-PE/Cy7 (clone S19004C), CD56-Brilliant Violet421 (clone HCD56), CD3-Brilliant Violet510 (clone Hit3a), CD337(NKp30)-Brilliant Violet605 (clone P30-15), CD335(NKp46)-Brilliant Violet650 (clone 9E2), CD57-Brilliant Violet711 (clone QA17A04), and CD16-Brilliant Violet785 (clone B73.1) (all from Biolegend) and Ghost Dye Red 780 (Tonbo Biosciences) prior to analysis by flow cytometry for UMAP analysis. To isolate CD4⁺ T cells, adherent macrophages were removed from PBMCs by culturing in flasks overnight in C10 (RPMI 1640 media supplemented with 10% vol/vol FBS (Omega Scientific), 1% L-glutamine, 1% penicillin/streptomycin (Invitrogen), 500 mM 2-mercaptoethanol (Sigma), 1 mM sodium pyruvate (Gibco), 0.1 mM MEM non-essential amino acids (Gibco), 10 mM Hepes (Gibco) and 20 ng per ml of recombinant human interleukin-2 (IL-2) (Peprotech). CD4⁺ T cells were isolated using CD4 MicroBeads (Miltenyi). 293T cell line was purchased from the American Type Culture Collection (ATCC) (ATCC CRL-11268). The following reagent was obtained through the NIH AIDS Reagent Program, Division of AIDS, NIAID, NIH: GHOST (3) CXCR4⁺CCR5+ cells from Dr. Vineet N. Kalamansi and Dr. Dan R. Littman (cat. #3942).

Transfections: To generate HIV-1 virus supernatant, plasmids were transfected into 293T cells in T-150 flasks, following manufacturer's protocol for the BioT transfection reagent (Bioland). Virus-containing supernatant was harvested at day 2 post-transfection and passed through a 0.45 μm filter. Aliquots of virus were frozen at −80° C. and thawed immediately prior to use.

Statistical analyses: Statistical analyses were performed using FlowJo v.10, Graphpad Prism 8.4.3, and Python 2.7.

Data availability: The data generated in this study are provided in the Supplementary Information/Source Data file. Source data are provided with this paper. The reads generated in this study have been deposited on SRA (Short Read Archive) database under the accession number (PRJNA694337).
Code availability: All codes are publicly available. See doi:10.5281/zenodo.5723824.

### NK and CD4⁺ T Cell Cocultures In Vitro

CD4⁺ T cells were cultured at 1×10⁶ cells per ml in C10 media with 20 ng per ml of IL-2 and Gibco Dynabeads Human T-Activator CD3/CD28 (Thermofisher) per manufacturer protocol for 3 days. Then co-stimulated CD4⁺ T cells were resuspended at 1.5×10⁶ cells per ml in C10 media with 20 ng per ml of IL-2 and spin-infected with 800 ng of p24 NL4-3 or NFNSX per 1×10⁶ cells at 1,200 g for 2 h at 25° C. The CD4⁺ T cells were washed twice with media and cocultured with NK cells at an effector-to-target (E:T) ratio of 1:1 overnight at 1×10⁶ cells per ml in C10 media with 20 ng per ml of IL-2. Flow cytometry was used to assess intracellular IFN-γ and CD107a levels at 24 h by staining with CD3 Pacific Blue (clone Hit3a), CD56-PE-Cy7 (clone HCD56), IFN-γ-PerCp (clone 4S.B3) and CD107a-PE (clone H4A3) (all from Biolegend), and Ghost Dye Red 780 (Tonbo Biosciences). Infection was also assessed at 48 h by staining with p24 using FITC-conjugated antibody (clone KC57) (Beckman Coulter).

### Barcode Analysis

RNA was extracted from the barcoded virus supernatant using QIAamp Viral RNA Mini Kit (Qiagen) and from cells using RNeasy MiniKit (Qiagen). cDNA was generated using SuperScript IV First-Strand Synthesis Kit (Invitrogen). The same amount of input RNA that was used for viral load measurement was also used for cDNA synthesis for barcode analysis. The barcode region was amplified by hemi-nested PCR using Phusion High-Fidelity DNA Polymerase (ThermoFisher Scientific). Each RNA molecule was tagged with a Primer ID using methods in the art. Primer ID removal and cDNA purification was performed using an Purelink Quick PCR Purification Kit (Invitrogen) and methods in the art. The amplified fragment was ligated with the sequencing adaptor, which had a six-nucleotide multiplexing ID to distinguish among different samples. Deep sequencing was performed with Illumina HiSeq3000 PE150. Sequencing depth was 10-fold higher than viral genome copies. Raw sequencing reads were de-multiplexed using the six-nucleotide ID. Sequencing error within the barcode region was corrected by filtering out low quality reads (quality score<30) and un-matched base-pairs between forward and reverse reads. PrimerIDs were used to correct sequencing errors. The frequency of Primer ID reads followed a bi-modal distribution (FIG. 53). The Primer IDs were filtered using the frequency cutoff between the Poisson distribution of errors and normal distribution of real Primer IDs. For each Primer ID, the most frequently observed barcode was called. Similar barcodes were then grouped into clusters. Clustered barcodes represent sequences with >4 bp differences from one another (FIG. 52). Barcode clusters with less than 400 occurrences were filtered to remove handling and sequencing errors. To prove the barcodes quantification are absolute number of clones, RNA molecules were sampled from the same population twice and found the number of barcodes is identical in the two samples (FIG. 54).

### In Vitro HIV-1 Infections

GHOST (3) CXCR4⁺CCR5+ cells were cultured in DMEM containing 10% vol/vol FBS, 500 μg per mL G418 (Gibco), 1% penicillin/streptomycin, 100 μg per mL hygromycin (Sigma), and 1 μg per mL puromycin (Sigma). Cells were seeded into 24-well tissue culture plates at 5×10⁴ cells per well. The next day media was replaced with various doses of HIV-1 and fresh media containing 10 μg per mL of polybrene (Sigma Aldrich). Plates were incubated for 2 h at 37° C. and then the media was replaced with 1 mL of fresh media without polybrene. Cells were incubated for a further 2 days, and then harvested by exposure to 0.05% trypsin (Gibco) in Phosphate Buffer Saline (PBS) (Gibco) for 5 min, and then removed by agitation with media. Cells were collected and fixed in 3% paraformaldehyde then analyzed for GFP expression by flow cytometry (data not shown). CD4⁺ T cells were isolated from PBMCs by immunomagnetic selection (Miltenyi) following the manufacturer's instructions, then were co-stimulated with Dynabead CD3/CD28 human T-activator (ThermoFisher Scientific) per manufacturer's instructions and cultured in C10 media containing 20 ng per ml of IL-2 (Peprotech). For infection, 5×10⁵ cells were exposed to HIV-1 in 200 μl of C10 media containing IL-2 and 10 μg per mL of polybrene. Cells were spin inoculated by centrifugation at 1,200 g for 1.5 h at 22° C. After spin-inoculation, cells were washed and resuspended in 200 μL of fresh C10 media containing IL-2. Viral infection was quantified by staining cells for p24 using PE-conjugated antibody to HIV core antigen (clone KC57) and analyzing by flow cytometry (data not shown). Cell-free supernatant samples were analyzed using HIV p24 enzyme-linked immunosorbent assay kit (Beckman Coulter).

### Mice

All mice were maintained in the animal facility at UCLA. All experiments were performed in ethical compliance with the study protocol approved by the UCLA Animal Research Committee (ARC #1996-058). Humanized bone marrow liver thymus (BLT) mice were constructed using methods in the art. In brief, NOD.Cg-Prkdc^{scid} I12rg^{tm1Wj1}/SzJ or NSG mice or C57BL/6 Rag2^{-/-}γ^{-/-}CD47^{-/-} or TKO mice⁵⁸ were obtained from Jackson Laboratories and bred at UCLA. Male and female mice were age-matched and between 6 and 8 weeks old were irradiated with 270 rads, and then pieces of fetal thymus and liver tissue were transplanted under the kidney capsule. Mice were then injected intravenously with 5×10⁴ human fetal liver-derived CD34+ cells isolated by immunomagnetic separation.

### Plasma Viral Load Measurements

For interval biweekly or weekly bleeds, 50 μl of blood was collected using EDTA-coated capillary tubes by retro-orbital bleed for viral load measurements. Whole blood was spun at 300 g for 5 min to separate plasma from the cellular fraction. Total RNA was extracted from plasma using QIAamp Viral RNA Minikit (Qiagen) per manufacturer's protocol. HIV-1 RNA was quantified by qRT-PCR. Reaction mixture was prepared using Taqman Fast Virus 1-Step Mastermix (ThermoFisher Scientific) with 20 μl eluted RNA and a sequence-specific targeting a conserved region of the HIV-1 gag gene probe using methods in the art. Cycle threshold values were calibrated using standard samples with known amounts of absolute plasmid DNA copies. The quantitation limit was determined to be 200 copies per ml. At necropsy, more than 100 μl of blood was collected from each mouse, which allowed at least 50 μl of plasma to be analyzed for barcode analysis.

### Tissue Harvest and Processing

Plasma was separated from blood as described above, and then remaining layer was lysed using RBC Lysis Buffer (Biolegend) to obtain the PBMCs. Splenocytes were obtained by passing disaggregated tissue through a 40 μm filter. Bone marrow cells were obtained by grinding bones using a mortar and pestle. Blood, spleen, and bone marrow cells were then fixed for flow cytometry as described below. Remaining cell pellets were stored for DNA analysis or suspended in RLT buffer (Qiagen) for RNA analysis and frozen at −80° C.

### Staining and Analysis for Flow Cytometry

Cells from animals were stained with fluorescently conjugated antibodies: CD69-Brilliant Violet 510 (clone FN50) or CD14-Brilliant Violet 510 (clone M5E2), CD3-Pacific Blue (clone Hit3a), CD8a-FITC (clone Hit8a), CD4-PE (clone RPA-T4), CD19-PE-Cy5 (clone SJ25C1), CD45-APC (clone 2D1), CD56-PE-Cy7 (clone MEM-188) (all from Biolegend) and Ghost Dye Red 780 (Tonbo Biosciences). All flow cytometry samples were run using a MACSQuant Analyzer 10 flow cytometer (Miltenyi) or Attune NxT. (Beckman Coulter). All data was analyzed using FlowJo v.10 (TreeStart, Inc).

### Cell-Associated (CA)-HIV RNA and Total HIV DNA

CA-HIV RNA was extracted from lysed splenocytes and bone marrow cells using RNeasy Minikit (Qiagen). CA-HIV DNA was extracted from cell pellets using DNeasy Blood & Tissue Kits (Qiagen). Viral loads were measured by qPCR using 500 ng of CA-HIV RNA and DNA with the same primers as above. CA-HIV RNA and DNA are reported as the number of HIV-1 RNA or DNA copies per 10⁶ CD4+ cells. The same amount of input RNA that was used for viral load measurement was used for PrimerID and barcode analysis. All cDNA was used as the template for the first round of PCR during the barcode analysis.

### Splenocyte Coculture

The spleens were aseptically collected from mice at the time of necropsy, passed through a 40 μm cell filter to achieve single-cell suspensions, and lysed using RBC Lysis Buffer (Biolegend). 2.5×10⁶ to 36×10⁶ splenocytes were resuspended in C10 media containing Piperacillin/tazobactam (Wockhardt), 2.5 μg per mL Amphotericin B (Fisher), and 20 ng per ml recombinant human IL-2 (Peprotech) at a concentration of 5×10⁵ to 2×10⁶ cells per ml. The splenocytes were cocultured with 1×10⁶ CEM cells and co-stimulated with 1 μg per ml anti-CD28 antibody (Tonbo biosciences; In Vivo Ready™ Anti-Human CD28 (clone CD28.2) on 6-well tissue culture plates pre-coated with 50 μg/ml goat anti-mouse antibody (Invitrogen; Goat anti-Mouse IgG (H+L) Secondary Antibody) and 1 μg per ml of OKT3 antibody (Tonbo biosciences; Purified Anti-Human CD3 (clone OKT3))⁴. 24 h after co-stimulation, the culture media were doubled. After 3 days of activation, the cells were resuspended in fresh media at 5×10⁵ cells per ml. For each subsequent passaging, the cells were cultured in a 17:3 ratio of fresh culture media and prior culture media at 5×10⁵ cells per ml. The supernatants were sampled on days 7, 10, and 14 days post co-stimulation and diluted in Triton X-100 (Sigma) in PBS at a final working concentration of 0.5% Triton-X. The samples were sent to the CFAR Virology Core at UCLA for HIV p24 antigen ELISA.

### Mathematical Modeling

To quantify the rate of virus spread following cessation of ART, a model of virus dynamics was used to track the temporal evolution of the free virus population, V, the infected cells population, I, and the susceptible target cell population, S. The model is given by the following set of ordinary differential equations (ODEs):

$$\frac{dS}{dt} = λ - dS - β SV \qquad ( 1 )$$

$$\frac{dI}{dt} = β SV - aI \qquad ( 2 )$$

$$\frac{dV}{dt} = kI - uV \qquad ( 3 )$$

Here in equations (1-3) λ is the total rate of susceptible target cell production and d is the (per cell) death rate of susceptible cells; β is infectivity (per virus), a is the per-cell death rate of infected cells, k is the per-cell rate of virus production, and u is the viral death rate; all rates measured in (days)⁻¹. The initial number of infected cells in the model corresponds to the infected cell population in which the virus has been activated following latency. Because the focus was only on the exponential phase of virus growth during rebound, the susceptible target cell population can be assumed to be constant. Assuming that the turnover of the virus population is significantly faster than that of the infected cells, the initial virus spread is described by the following ODE (4):

$$\frac{dI}{dt} = {β}^{′} I - aI \qquad ( 4 )$$

where β′=βkS/u and the viral spread rate is given by r=β′−α.

The details of the fitting procedure and statistical analysis are as follows. To obtain the best fit for the viral spread rate, r, in the absence and in the presence of NK cell treatment, standard linear regression was used to fit the post-ART growth data across all experimental repeats for each of the two conditions (FIG. 50), which yielded the estimate for the slopes. In order to evaluate whether the viral spread rate was different in the absence and in the presence of NK cells, z-statistics were used for the difference in regression slopes. It was found that the viral spread rates are significantly different in mice that were and were not treated with NK cells (p=0.001).

### REFERENCES

The following references are herein incorporated by reference in their entirety with the exception that, should the scope and meaning of a term conflict with a definition explicitly set forth herein, the definition explicitly set forth herein controls:
Kim J T, et al. Latency reversal plus natural killer cells diminish HIV reservoir in vivo. Nat Commun. 2022 Jan. 10; 13(1):121. doi: 10.1038/s41467-021-27647-0. PMID: 35013215; PMCID: PMC8748509.
Davey, R. T., Jr. et al. HIV-1 and T cell dynamics after interruption of highly active antiretroviral therapy (HAART) in patients with a history of sustained viral suppression. Proc Natl Acad Sci USA 96, 15109-15114, doi:10.1073/pnas.96.26.15109 (1999).
Wong, J. K. et al. Recovery of replication-competent HIV despite prolonged suppression of plasma viremia. Science 278, 1291-1295, doi:10.1126/science.278.5341.1291 (1997).
Chun, T. W., Davey, R. T., Jr., Engel, D., Lane, H. C. & Fauci, A. S. Re-emergence of HIV after stopping therapy. Nature 401, 874-875, doi:10.1038/44755 (1999).
Marsden, M. D. & Zack, J. A. Experimental Approaches for Eliminating Latent HIV. For Immunopathol Dis Therap 6, 91-99, doi:10.1615/forumimmundisther.2016015242 (2015).
Thorlund, K., Horwitz, M. S., Fife, B. T., Lester, R. & Cameron, D. W. Landscape review of current HIV ‘kick and kill’ cure research—some kicking, not enough killing. Bmc Infect Dis 17, doi:ARTN 595
10.1186/s12879-017-2683-3 (2017).
Marsden, M. D. et al. In vivo activation of latent HIV with a synthetic bryostatin analog effects both latent cell “kick” and “kill” in strategy for virus eradication. PLoS Pathog 13, e1006575, doi:10.1371/journal.ppat.1006575 (2017).
DeChristopher, B. A. et al. Designed, synthetically accessible bryostatin analogues potently induce activation of latent HIV reservoirs in vitro. Nat Chem 4, 705-710, doi:10.1038/Nchem.1395 (2012).
Marsden, M. D. et al. Tracking HIV Rebound following Latency Reversal Using Barcoded HIV. Cell Rep Med 1, 100162, doi:10.1016/j.xcrm.2020.100162 (2020).
Ward, A. R., Mota, T. M. & Jones, R. B. Immunological approaches to HIV cure. Semin Immunol, 101412, doi:10.1016/j.smim.2020.101412 (2020).
Halper-Stromberg, A. et al. Broadly neutralizing antibodies and viral inducers decrease rebound from HIV-1 latent reservoirs in humanized mice. Cell 158, 989-999, doi:10.1016/j.cell.2014.07.043 (2014).
Borducchi, E. N. et al. Ad26/MVA therapeutic vaccination with TLR7 stimulation in SIV-infected rhesus monkeys. Nature 540, 284-287, doi:10.1038/nature20583 (2016).
Borducchi, E. N. et al. Antibody and TLR7 agonist delay viral rebound in SHIV-infected monkeys. Nature 563, 360-364, doi:10.1038/s41586-018-0600-6 (2018).
Scott-Algara, D. et al. Cutting edge: increased NK cell activity in HIV-1-exposed but uninfected Vietnamese intravascular drug users. J Immunol 171, 5663-5667, doi:10.4049/jimmunol.171.11.5663 (2003).
Flores-Villanueva, P. O. et al. Control of HIV-1 viremia and protection from AIDS are associated with HLA-Bw4 homozygosity. Proc Natl Acad Sci USA 98, 5140-5145, doi:10.1073/pnas.071548198 (2001).
Gondois-Rey, F. et al. NKG2C(+) memory-like NK cells contribute to the control of HIV viremia during primary infection: Optiprim-ANRS 147. Clin Transl Immunology 6, e150, doi:10.1038/cti.2017.22 (2017).
Ma, M. et al. NKG2C(+)NKG2A(−) Natural Killer Cells are Associated with a Lower Viral Set Point and may Predict Disease Progression in Individuals with Primary HIV Infection. Front Immunol 8, 1176, doi:10.3389/fimmu.2017.01176 (2017).
Martin, M. P. et al. Epistatic interaction between KIR3DS1 and HLA-B delays the progression to AIDS. Nat Genet 31, 429-434, doi:10.1038/ng934 (2002).
Alter, G. et al. HIV-1 adaptation to NK-cell-mediated immune pressure. Nature 476, 96-100, doi:10.1038/nature10237 (2011).
Holzemer, A. et al. Selection of an HLA-C*03:04-Restricted HIV-1 p24 Gag Sequence Variant Is Associated with Viral Escape from KIR2DL3+Natural Killer Cells: Data from an Observational Cohort in South Africa. PLoS Med 12, e1001900; discussion e1001900, doi:10.1371/journal.pmed.1001900 (2015).
Giuliani, E. et al. NK cells of HIV-1-infected patients with poor CD4(+) T-cell reconstitution despite suppressive HAART show reduced IFN-gamma production and high frequency of autoreactive CD56(bright) cells. Immunol Lett 190, 185-193, doi:10.1016/j.imlet.2017.08.014 (2017).
Mavilio, D. et al. Characterization of CD56−/CD16+ natural killer (NK) cells: a highly dysfunctional NK subset expanded in HIV-infected viremic individuals. Proc Natl Acad Sci USA 102, 2886-2891, doi:10.1073/pnas.0409872102 (2005).
Goodier, M. R., Imami, N., Moyle, G., Gazzard, B. & Gotch, F. Loss of the CD56hiCD16− NK cell subset and NK cell interferon-gamma production during antiretroviral therapy for HIV-1: partial recovery by human growth hormone. Clin Exp Immunol 134, 470-476, doi:10.1111/j.1365-2249.2003.02329.x (2003).
Migueles, S. A. et al. Defective human immunodeficiency virus-specific CD8+ T-cell polyfunctionality, proliferation, and cytotoxicity are not restored by antiretroviral therapy. J Virol 83, 11876-11889, doi:10.1128/JVI.01153-09 (2009).
Jennes, W. et al. Inhibitory KIR/HLA incompatibility between sexual partners confers protection against HIV-1 transmission. Blood 121, 1157-1164, doi:10.1182/blood-2012-09-455352 (2013).
Ni, Z. Y., Knorr, D. A., Bendzick, L., Allred, J. & Kaufman, D. S. Expression of Chimeric Receptor CD4 zeta by Natural Killer Cells Derived from Human Pluripotent Stem Cells Improves In Vitro Activity but Does Not Enhance Suppression of HIV Infection In Vivo. Stem Cells 32, 1021-1031, doi:10.1002/stem.1611 (2014).
Garrido, C. et al. Interleukin-15-Stimulated Natural Killer Cells Clear HIV-1-Infected Cells following Latency Reversal Ex Vivo. J Virol 92, doi:10.1128/JVI.00235-18 (2018).
Cooper, M. A., Fehniger, T. A. & Caligiuri, M. A. The biology of human natural killer-cell subsets. Trends Immunol 22, 633-640, doi:10.1016/s1471-4906(01)02060-9 (2001).
Nagler, A., Lanier, L. L., Cwirla, S. & Phillips, J. H. Comparative studies of human FcRIII-positive and negative natural killer cells. J Immunol 143, 3183-3191 (1989).
Angelo, L. S. et al. Practical NK cell phenotyping and variability in healthy adults. Immunol Res 62, 341-356, doi:10.1007/s12026-015-8664-y (2015).
Thielens, A., Vivier, E. & Romagne, F. NK cell MHC class I specific receptors (KIR): from biology to clinical intervention. Curr Opin Immunol 24, 239-245, doi:10.1016/j.coi.2012.01.001 (2012).
Beziat, V. et al. CD56brightCD16+NK cells: a functional intermediate stage of NK cell differentiation. J Immunol 186, 6753-6761, doi:10.4049/jimmunol.1100330 (2011).
O'Brien, W. A. et al. HIV-1 tropism for mononuclear phagocytes can be determined by regions of gp120 outside the CD4-binding domain. Nature 348, 69-73, doi:10.1038/348069a0 (1990).
Ni, Z. et al. Human pluripotent stem cells produce natural killer cells that mediate anti-HIV-1 activity by utilizing diverse cellular mechanisms. J Virol 85, 43-50, doi:10.1128/JVI.01774-10 (2011).
Li, Y., Hermanson, D. L., Moriarity, B. S. & Kaufman, D. S. Human iPSC-Derived Natural Killer Cells Engineered with Chimeric Antigen Receptors Enhance Anti-tumor Activity. Cell Stem Cell 23, 181-192 e185, doi:10.1016/j.stem.2018.06.002 (2018).
Denton, P. W. et al. Generation of HIV latency in humanized BLT mice. J Virol 86, 630-634, doi:10.1128/JVI.06120-11 (2012).
Melkus, M. W. et al. Humanized mice mount specific adaptive and innate immune responses to EBV and TSST-1. Nat Med 12, 1316-1322, doi:10.1038/nm1431 (2006).
Huntington, N. D. et al. IL-15 trans-presentation promotes human NK cell development and differentiation in vivo. J Exp Med 206, 25-34, doi:10.1084/jem.20082013 (2009).
Vahedi, F. et al. Ex Vivo Expanded Human NK Cells Survive and Proliferate in Humanized Mice with Autologous Human Immune Cells. Sci Rep 7, 12083, doi:10.1038/s41598-017-12223-8 (2017).
Nikzad, R. et al. Human natural killer cells mediate adaptive immunity to viral antigens. Sci Immunol 4, doi:10.1126/sciimmunol.aat8116 (2019).
Vogel, B., Tennert, K., Full, F. & Ensser, A. Efficient generation of human natural killer cell lines by viral transformation. Leukemia 28, 192-195, doi:10.1038/leu.2013.188 (2014).
Zhang, Y. et al. In vivo kinetics of human natural killer cells: the effects of ageing and acute and chronic viral infection. Immunology 121, 258-265, doi:10.1111/j.1365-2567.2007.02573.x (2007).
Ruggeri, L. et al. Effectiveness of donor natural killer cell alloreactivity in mismatched hematopoietic transplants. Science 295, 2097-2100, doi:10.1126/science.1068440 (2002).
Curti, A. et al. Successful transfer of alloreactive haploidentical KIR ligand-mismatched natural killer cells after infusion in elderly high risk acute myeloid leukemia patients. Blood 118, 3273-3279, doi:10.1182/blood-2011-01-329508 (2011).
Rubnitz, J. E. et al. NKAML: a pilot study to determine the safety and feasibility of haploidentical natural killer cell transplantation in childhood acute myeloid leukemia. J Clin Oncol 28, 955-959, doi:10.1200/JCO.2009.24.4590 (2010).
Fennessey, C. M. et al. Genetically-barcoded SIV facilitates enumeration of rebound variants and estimation of reactivation rates in nonhuman primates following interruption of suppressive antiretroviral therapy. PLoS Pathog 13, e1006359, doi:10.1371/journal.ppat.1006359 (2017).
Ali, A. & Yang, O. O. A novel small reporter gene and HIV-1 fitness assay. J Virol Methods 133, 41-47, doi:10.1016/j.jviromet.2005.10.016 (2006).
Nowak, M. M., Robert. Virus Dynamics: Mathematical Principles of Immunology And Virology. (Oxford University Press, 2001).
Perelson, A. S. Modelling viral and immune system dynamics. Nat Rev Immunol 2, 28-36, doi:10.1038/nri700 (2002).
Nowak, M. A. & May, R. M. Virus dynamics: mathematical principles of immunology and virology. (Oxford University Press, 2000).
Jabara, C. B., Jones, C. D., Roach, J., Anderson, J. A. & Swanstrom, R. Accurate sampling and deep sequencing of the HIV-1 protease gene using a Primer ID. Proc Natl Acad Sci USA 108, 20166-20171, doi:10.1073/pnas.1110064108 (2011).
Zhou, S., Jones, C., Mieczkowski, P. & Swanstrom, R. Primer ID Validates Template Sampling Depth and Greatly Reduces the Error Rate of Next-Generation Sequencing of HIV-1 Genomic RNA Populations. J Virol 89, 8540-8555, doi:10.1128/JVI.00522-15 (2015).
Marsden, M. D. et al. Characterization of designed, synthetically accessible bryostatin analog HIV latency reversing agents. Virology 520, 83-93, doi:10.1016/j.virol.2018.05.006 (2018).
Lavender, K. J. et al. BLT-humanized C57BL/6 Rag2−/−gammac−/−CD47−/− mice are resistant to GVHD and develop B- and T-cell immunity to HIV infection. Blood 122, 4013-4020, doi:10.1182/blood-2013-06-506949 (2013).
Davis, Z. B., Felices, M., Verneris, M. R. & Miller, J. S. Natural Killer Cell Adoptive Transfer Therapy: Exploiting the First Line of Defense Against Cancer. Cancer J 21, 486-491, doi:10.1097/PPO.0000000000000156 (2015).
Jeyaraman, M. et al. Bracing NK cell based therapy to relegate pulmonary inflammation in COVID-19. Heliyon 7, e07635, doi:10.1016/j.heliyon.2021.e07635 (2021).
Schaufelberger, D. E. et al. The large-scale isolation of bryostatin 1 from Bugula neritina following current good manufacturing practices. J Nat Prod 54, 1265-1270, doi:10.1021/np50077a004 (1991).
Wender, P. A. et al. Scalable synthesis of bryostatin 1 and analogs, adjuvant leads against latent HIV. Science 358, 218-223, doi:10.1126/science.aan7969 (2017).
Lavender, K. J. et al. An advanced BLT-humanized mouse model for extended HIV-1 cure studies. AIDS 32, 1-10, doi:10.1097/QAD.0000000000001674 (2018).
Richard, J., Sindhu, S., Pham, T. N. Q., Belzile, J. P. & Cohen, E. A. HIV-1 Vpr up-regulates expression of ligands for the activating NKG2D receptor and promotes NK cell-mediated killing. Blood 115, 1354-1363, doi:10.1182/blood-2009-08-237370 (2010).
Ward, J. et al. HIV-1 Vpr Triggers Natural Killer Cell-Mediated Lysis of Infected Cells through Activation of the ATR-Mediated DNA Damage Response. Plos Pathogens 5, doi:ARTN e100061310.1371/journal.ppat.1000613 (2009).
Pache, L. et al. Pharmacological Activation of Non-canonical NF-kappaB Signaling Activates Latent HIV-1 Reservoirs In Vivo. Cell Rep Med 1, 100037, doi:10.1016/j.xcrm.2020.100037 (2020).

Perelson, A. S. Modelling viral and immune system dynamics. Nature Rev Immunol 2, 28-36. (2002).

Nowak, M. A. & May, R. M. Virus dynamics. Mathematical principles of immunology and virology., (Oxford University Press, 2000).

Cohen, J., Cohen, P., West, S. G. & Aiken, L. S. Applied Multiple Regression/Correlation Analysis for the Behavioral Sciences (3rd edition). (Lawrence Earlbaum Associates, 2003).

All scientific and technical terms used in this application have meanings commonly used in the art unless otherwise specified.

As used herein, a “bryostatin compound” comprises the following backbone structure as part of its chemical structure:

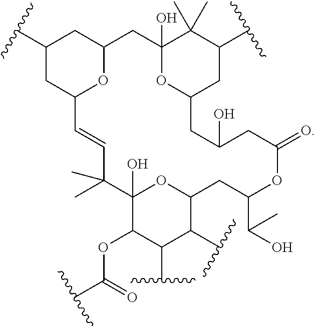 [SMILES: *C(=O)OC1C(*)C(*)C2CC(C(C)O)OC(=O)CC(O)CC3CC(*)C(C)(C)C(O)(CC4CC(*)CC(/C=C/C(C)(C)C1(O)O2)O4)O3]

As used herein, the terms “subject”, “patient”, and “individual” are used interchangeably to refer to humans and non-human animals. The terms “non-human animal” and “animal” refer to all non-human vertebrates, e.g., non-human mammals and non-mammals, such as non-human primates, horses, sheep, dogs, cows, pigs, chickens, and other veterinary subjects and test animals. In some embodiments, the subject is a mammal. In some embodiments, the subject is a human.

The use of the singular can include the plural unless specifically stated otherwise. As used in the specification and the appended claims, the singular forms “a”, “an”, and “the” can include plural referents unless the context clearly dictates otherwise.

As used herein, “and/or” means “and” or “or”. For example, “A and/or B” means “A, B, or both A and B” and “A, B, C, and/or D” means “A, B, C, D, or a combination thereof” and said “A, B, C, D, or a combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

As used herein, the phrase “one or more of”, e.g., “one or more of A, B, and/or C” means “one or more of A”, “one or more of B”, “one or more of C”, “one or more of A and one or more of B”, “one or more of B and one or more of C”, “one or more of A and one or more of C” and “one or more of A, one or more of B, and one or more of C”.

As used herein, the phrase “consists essentially of” in the context of a given ingredient in a composition, means that the composition may include additional ingredients so long as the additional ingredients do not adversely impact the activity, e.g., biological or pharmaceutical function, of the given ingredient. In the context of compositions, “consists essentially of” means that the composition may comprise additional ingredients so long as the additional ingredients do not adversely impact the efficacy of the recited active ingredient(s), e.g., NK cells and the one or more LRAs. In the context of treatment methods, “consists essentially of” means that the methods may comprise additional steps so long as the additional steps do not adversely impact the efficacy of the administration of NK cells and the one or more LRAs.

The phrase “comprises, consists essentially of, or consists of A” is used as a tool to avoid excess page and translation fees and means that in some embodiments the given thing at issue: comprises A, consists essentially of A, or consists of A. For example, the sentence “In some embodiments, the composition comprises, consists essentially of, or consists of A” is to be interpreted as if written as the following three separate sentences: “In some embodiments, the composition comprises A. In some embodiments, the composition consists essentially of A. In some embodiments, the composition consists of A.”

Similarly, a sentence reciting a string of alternates is to be interpreted as if a string of sentences were provided such that each given alternate was provided in a sentence by itself. For example, the sentence “In some embodiments, the composition comprises A, B, or C” is to be interpreted as if written as the following three separate sentences: “In some embodiments, the composition comprises A. In some embodiments, the composition comprises B. In some embodiments, the composition comprises C.” As another example, the sentence “In some embodiments, the composition comprises at least A, B, or C” is to be interpreted as if written as the following three separate sentences: “In some embodiments, the composition comprises at least A. In some embodiments, the composition comprises at least B. In some embodiments, the composition comprises at least C.”

### Additional Embodiments

Embodiment 1. A composition, kit, or combination product for reducing an amount of a human immunodeficiency virus (HIV) in a subject, which comprises, consists essentially of, or consists of a bryostatin compound in combination with natural killer (NK) cells.

Embodiment 2. The composition, the kit, or the combination product according to Embodiment 1, wherein the bryostatin compound, is SUW133.

Embodiment 3. The composition, the kit, or the combination product according to Embodiment 1 or Embodiment 2, wherein the NK cells are CD56⁺CD3⁻ NK cells.

Embodiment 4. The composition, the kit, or the combination product according to any one of Embodiments 1-3, wherein the NK cells are human peripheral blood NK cells.

Embodiment 5. The composition, the kit, or the combination product according to any one of Embodiments 1-4, wherein the composition, the kit, or the combination product comprises
a) about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, or about 120 mg of the bryostatin compound; and
b) about 60×10⁶, about 90×10⁶, about 120×10⁶, about 150×10⁶, about 180×10⁶, about 210×10⁶, about 240×10⁶, about 270×10⁶, about 300×10⁶, about 330×10⁶, about 360×10⁶, about 390×10⁶, about 420×10⁶, about 450×10⁶, about 480×10⁶, about 510×10⁶, about 540×10⁶, about 570×10⁶, about 600×10⁶, about 630×10⁶, about 660×10⁶, about 690×10⁶, about 720×10⁶, about 750×10⁶, about 780×10⁶, about 810×10⁶, about 840×10⁶, about 870×10⁶, about 900×10⁶, about 930×10⁶, about 960×10⁶, about 990×10⁶, about 1020×10⁶, about 1050×10⁶, about 1080×10⁶, about 1110×10⁶, about 1140×10⁶, about 1170×10⁶, about 1200×10⁶, about 1230×10⁶, about 1260×10⁶, about 1290×10⁶, about 1320×10⁶, about 1350×10⁶, about 1380×10⁶, about 1410×10⁶, about 1440×10⁶, about 1470×10⁶, about 1500×10⁶, about 1530×10⁶, about 1560×10⁶, about 1590×10⁶, about 1620×10⁶, about 1650×10⁶, about 1680×10⁶, about 1710×10⁶, about 1740×10⁶, about 1770×10⁶, or about 1800×10⁶ of the NK cells.

Embodiment 6. A method of reducing an amount of a human immunodeficiency virus (HIV) in a subject, which comprises, consists essentially of, or consists of administering to the subject a bryostatin compound and NK cells.

Embodiment 7. The method according to Embodiment 6, wherein the bryostatin compound, is SUW133.

Embodiment 8. The method according to Embodiment 6 or Embodiment 7, wherein the NK cells are CD56⁺CD3⁻ NK cells.

Embodiment 9. The method according to any one of Embodiments 6-8, wherein the NK cells are human peripheral blood NK cells.

Embodiment 10. The method according to any one of Embodiments 6-9, wherein the NK cells are administered about 2-14 hours, about 4-12 hours, about 6-10 hours, or about 8 hours after administration of the bryostatin compound.

Embodiment 11. The method according to any one of Embodiments 6-10, wherein the bryostatin compound is administered 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, or 24 days after ART cessation.

Embodiment 12. The method according to any one of Embodiments 6-11, wherein the amount of the bryostatin compound administered to the subject is
a) about 0.75-2, about 1.0-1.75, about 1.1-1.3, about 1.25 mg/kg body weight of the subject; or
b) about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, or about 120 mg.

Embodiment 13. The method according to any one of Embodiments 6-12, wherein the amount of the NK cells administered to the subject is
a) about 1-30×10⁶, about 10-30×10⁶, or about 25×10⁶ of NK cells/kg body weight of the subject; or
b) about 60×10⁶, about 90×10⁶, about 120×10⁶, about 150×10⁶, about 180×10⁶, about 210×10⁶, about 240×10⁶, about 270×10⁶, about 300×10⁶, about 330×10⁶, about 360×10⁶, about 390×10⁶, about 420×10⁶, about 450×10⁶, about 480×10⁶, about 510×10⁶, about 540×10⁶, about 570×10⁶, about 600×10⁶, about 630×10⁶, about 660×10⁶, about 690×10⁶, about 720×10⁶, about 750×10⁶, about 780×10⁶, about 810×10⁶, about 840×10⁶, about 870×10⁶, about 900×10⁶, about 930×10⁶, about 960×10⁶, about 990×10⁶, about 1020×10⁶, about 1050×10⁶, about 1080×10⁶, about 1110×10⁶, about 1140×10⁶, about 1170×10⁶, about 1200×10⁶, about 1230×10⁶, about 1260×10⁶, about 1290×10⁶, about 1320×10⁶, about 1350×10⁶, about 1380×10⁶, about 1410×10⁶, about 1440×10⁶, about 1470×10⁶, about 1500×10⁶, about 1530×10⁶, about 1560×10⁶, about 1590×10⁶, about 1620×10⁶, about 1650×10⁶, about 1680×10⁶, about 1710×10⁶, about 1740×10⁶, about 1770×10⁶, or about 1800×10⁶.

Embodiment 14. The method according to any one of Embodiments 6-11, wherein
a) the amount of the bryostatin compound administered to the subject is about 0.75-2, about 1.0-1.75, about 1.1-1.3, about 1.25 mg/kg body weight of the subject; and
b) the amount of the NK cells administered to the subject is about 1-30×10⁶, about 10-30×10⁶, or about 25×10⁶ NK cells/kg body weight of the subject.

Embodiment 15. The method according to any one of Embodiments 6-11, wherein
a) the amount of the bryostatin compound administered to the subject is about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, or about 120 mg; and
b) the amount of the NK cells administered to the subject is about 60×10⁶, about 90×10⁶, about 120×10⁶, about 150×10⁶, about 180×10⁶, about 210×10⁶, about 240×10⁶, about 270×10⁶, about 300×10⁶, about 330×10⁶, about 360×10⁶, about 390×10⁶, about 420×10⁶, about 450×10⁶, about 480×10⁶, about 510×10⁶, about 540×10⁶, about 570×10⁶, about 600×10⁶, about 630×10⁶, about 660×10⁶, about 690×10⁶, about 720×10⁶, about 750×10⁶, about 780×10⁶, about 810×10⁶, about 840×10⁶, about 870×10⁶, about 900×10⁶, about 930×10⁶, about 960×10⁶, about 990×10⁶, about 1020×10⁶, about 1050×10⁶, about 1080×10⁶, about 1110×10⁶, about 1140×10⁶, about 1170×10⁶, about 1200×10⁶, about 1230×10⁶, about 1260×10⁶, about 1290×10⁶, about 1320×10⁶, about 1350×10⁶, about 1380×10⁶, about 1410×10⁶, about 1440×10⁶, about 1470×10⁶, about 1500×10⁶, about 1530×10⁶, about 1560×10⁶, about 1590×10⁶, about 1620×10⁶, about 1650×10⁶, about 1680×10⁶, about 1710×10⁶, about 1740×10⁶, about 1770×10⁶, or about 1800×10⁶.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

What is claimed is:

1. A method of reducing an amount of a human immunodeficiency virus (HIV) in a subject, which comprises, consists essentially of, or consists of administering to the subject a bryostatin compound and NK cells.

2. The method according to claim 1, wherein the bryostatin compound, is SUW133.

3. The method according to claim 1, wherein the NK cells are CD56⁺CD3⁻ NK cells.

4. The method according to claim 1, wherein the NK cells are human peripheral blood NK cells.

5. The method according to claim 1, wherein the NK cells are administered about 2-14 hours, about 4-12 hours, about 6-10 hours, or about 8 hours after administration of the bryostatin compound.

6. The method according to claim 1, wherein the bryostatin compound is administered 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, or 24 days after ART cessation.

7. The method according to claim 1, wherein the amount of the bryostatin compound administered to the subject is
a) about 0.75-2, about 1.0-1.75, about 1.1-1.3, about 1.25 mg/kg body weight of the subject; or
b) about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, or about 120 mg.

8. The method according to claim 1, wherein the amount of the NK cells administered to the subject is
a) about 1-30×10⁶, about 10-30×10⁶, or about 25×10⁶ of NK cells/kg body weight of the subject; or
b) about 60×10⁶, about 90×10⁶, about 120×10⁶, about 150×10⁶, about 180×10⁶, about 210×10⁶, about 240×10⁶, about 270×10⁶, about 300×10⁶, about 330×10⁶, about 360×10⁶, about 390×10⁶, about 420×10⁶, about 450×10⁶, about 480×10⁶, about 510×10⁶, about 540×10⁶, about 570×10⁶, about 600×10⁶, about 630×10⁶, about 660×10⁶, about 690×10⁶, about 720×10⁶, about 750×10⁶, about 780×10⁶, about 810×10⁶, about 840×10⁶, about 870×10⁶, about 900×10⁶, about 930×10⁶, about 960×10⁶, about 990×10⁶, about 1020×10⁶, about 1050×10⁶, about 1080×10⁶, about 1110×10⁶, about 1140×10⁶, about 1170×10⁶, about 1200×10⁶, about 1230×10⁶, about 1260×10⁶, about 1290×10⁶, about 1320×10⁶, about 1350×10⁶, about 1380×10⁶, about 1410×10⁶, about 1440×10⁶, about 1470×10⁶, about 1500×10⁶, about 1530×10⁶, about 1560×10⁶, about 1590×10⁶, about 1620×10⁶, about 1650×10⁶, about 1680×10⁶, about 1710×10⁶, about 1740×10⁶, about 1770×10⁶, or about 1800×10⁶.

9. The method according to claim 1, wherein
a) the amount of the bryostatin compound administered to the subject is about 0.75-2, about 1.0-1.75, about 1.1-1.3, about 1.25 mg/kg body weight of the subject; and
b) the amount of the NK cells administered to the subject is about 1-30×10⁶, about 10-30×10⁶, or about 25×10⁶ NK cells/kg body weight of the subject.

10. The method according to claim 1, wherein
a) the amount of the bryostatin compound administered to the subject is about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, or about 120 mg; and
b) the amount of the NK cells administered to the subject is about 60×10⁶, about 90×10⁶, about 120×10⁶, about 150×10⁶, about 180×10⁶, about 210×10⁶, about 240×10⁶, about 270×10⁶, about 300×10⁶, about 330×10⁶, about 360×10⁶, about 390×10⁶, about 420×10⁶, about 450×10⁶, about 480×10⁶, about 510×10⁶, about 540×10⁶, about 570×10⁶, about 600×10⁶, about 630×10⁶, about 660×10⁶, about 690×10⁶, about 720×10⁶, about 750×10⁶, about 780×10⁶, about 810×10⁶, about 840×10⁶, about 870×10⁶, about 900×10⁶, about 930×10⁶, about 960×10⁶, about 990×10⁶, about 1020×10⁶, about 1050×10⁶, about 1080×10⁶, about 1110×10⁶, about 1140×10⁶, about 1170×10⁶, about 1200×10⁶, about 1230×10⁶, about 1260×10⁶, about 1290×10⁶, about 1320×10⁶, about 1350×10⁶, about 1380×10⁶, about 1410×10⁶, about 1440×10⁶, about 1470×10⁶, about 1500×10⁶, about 1530×10⁶, about 1560×10⁶, about 1590×10⁶, about 1620×10⁶, about 1650×10⁶, about 1680×10⁶, about 1710×10⁶, about 1740×10⁶, about 1770×10⁶, or about 1800×10⁶.

11. A composition for reducing an amount of a human immunodeficiency virus (HIV) in a subject, which comprises, consists essentially of, or consists of a bryostatin compound in combination with natural killer (NK) cells.

12. The composition according to claim 11, wherein the bryostatin compound, is SUW133.

13. The composition according to claim 11, wherein the NK cells are CD56⁺CD3⁻ NK cells.

14. The composition according to claim 11, wherein the NK cells are human peripheral blood NK cells.

15. The composition according to claim 11, wherein the composition comprises
a) about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, or about 120 mg of the bryostatin compound; and
b) about 60×10⁶, about 90×10⁶, about 120×10⁶, about 150×10⁶, about 180×10⁶, about 210×10⁶, about 240×10⁶, about 270×10⁶, about 300×10⁶, about 330×10⁶, about 360×10⁶, about 390×10⁶, about 420×10⁶, about 450×10⁶, about 480×10⁶, about 510×10⁶, about 540×10⁶, about 570×10⁶, about 600×10⁶, about 630×10⁶, about 660×10⁶, about 690×10⁶, about 720×10⁶, about 750×10⁶, about 780×10⁶, about 810×10⁶, about 840×10⁶, about 870×10⁶, about 900×10⁶, about 930×10⁶, about 960×10⁶, about 990×10⁶, about 1020×10⁶, about 1050×10⁶, about 1080×10⁶, about 1110×10⁶, about 1140×10⁶, about 1170×10⁶, about 1200×10⁶, about 1230×10⁶, about 1260×10⁶, about 1290×10⁶, about 1320×10⁶, about 1350×10⁶, about 1380×10⁶, about 1410×10⁶, about 1440×10⁶, about 1470×10⁶, about 1500×10⁶, about 1530×10⁶, about 1560×10⁶, about 1590×10⁶, about 1620×10⁶, about 1650×10⁶, about 1680×10⁶, about 1710×10⁶, about 1740×10⁶, about 1770×10⁶, or about 1800×10⁶ of the NK cells.

16. A kit for reducing an amount of a human immunodeficiency virus (HIV) in a subject, which comprises, consists essentially of, or consists of a bryostatin compound packaged together with natural killer (NK) cells.

17. The kit according to claim 11, wherein the bryostatin compound, is SUW133.

18. The kit according to claim 11, wherein the NK cells are CD56⁺CD3⁻ NK cells.

19. The kit according to claim 11, wherein the NK cells are human peripheral blood NK cells.

20. The kit according to claim 11, wherein the kit comprises
a) about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, or about 120 mg of the bryostatin compound; and
b) about 60×10⁶, about 90×10⁶, about 120×10⁶, about 150×10⁶, about 180×10⁶, about 210×10⁶, about 240×10⁶, about 270×10⁶, about 300×10⁶, about 330×10⁶, about 360×10⁶, about 390×10⁶, about 420×10⁶, about 450×10⁶, about 480×10⁶, about 510×10⁶, about 540×10⁶, about 570×10⁶, about 600×10⁶, about 630×10⁶, about 660×10⁶, about 690×10⁶, about 720×10⁶, about 750×10⁶, about 780×10⁶, about 810×10⁶, about 840×10⁶, about 870×10⁶, about 900×10⁶, about 930×10⁶, about 960×10⁶, about 990×10⁶, about 1020×10⁶, about 1050×10⁶, about 1080×10⁶, about 1110×10⁶, about 1140×10⁶, about 1170×10⁶, about 1200×10⁶, about 1230×10⁶, about 1260×10⁶, about 1290×10⁶, about 1320×10⁶, about 1350×10⁶, about 1380×10⁶, about 1410×10⁶, about 1440×10⁶, about 1470×10⁶, about 1500×10⁶, about 1530×10⁶, about 1560×10⁶, about 1590×10⁶, about 1620×10⁶, about 1650×10⁶, about 1680×10⁶, about 1710×10⁶, about 1740×10⁶, about 1770×10⁶, or about 1800×10⁶ of the NK cells.
